(19) United States
(12) Patent Application Publication
(10) Pub. No.: US 2007/0265187 A1
(43) Pub. Date: Nov. 15, 2007

(54) Oral Formulations Comprising Bone Morphogenetic Proteins For Treating Metabolic Bone Diseases

(76) Inventors: Slobodan Vukicevic, Zagreb (HR); Petra Simic, Zagreb (HR); Hermann Oppermann, Medway, MA (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/568,247

(22) PCT Filed: Apr. 28, 2005
(86) PCT No.: PCT/US05/14410

§ 371 Date: Oct. 24, 2006

Related U.S. Application Data
(60) Provisional application No. 60/566,242, filed on Apr. 29, 2004.

(51) Int. Cl. A61K 38/02; A61P 19/10

(52) U.S. Cl. 514/2

(57) ABSTRACT

Methods and formulations for the administration of a bone morphogenetic protein (BMP) anywhere along the alimentary canal of an individual are described for use in treating osteoporosis or other metabolic bone diseases.

### CROSS-REFERENCE TO RELATED APPLICATIONS

This application claims priority to U.S. Provisional Application No. 60/566,242, filed Apr. 29, 2004.

### FIELD OF THE INVENTION

This invention is generally in the field of formulations for oral administration of therapeutic proteins. In particular, the invention provides formulations comprising bone morphogenetic proteins for use in treating metabolic diseases such as osteoporosis and other metabolic bone diseases.

### BACKGROUND OF THE INVENTION

Osteoporosis is a systemic skeletal disease that is characterized by low bone mass and deterioration of bone tissue with a consequent increase in bone fragility and susceptibility to fracture. It is the most common type of metabolic bone disease in the United States, where the condition affects more than 25 million people. The disease causes more than 1.3 million fractures each year, including 500,000 spine fractures, 250,000 hip fractures, and 240,000 wrist fractures. Hip fractures are the most serious consequence of osteoporosis, with 5%-20% of patients dying within one year, and over 50% of survivors being incapacitated.

Osteoporosis literally means “porous bones”. Healthy bones in the skeleton have a thick outer shelf and a strong inner mesh filled with collagen (protein), calcium salts, and other minerals. The inside of a healthy bone has the appearance of a honeycomb or network of bone with blood vessels and bone marrow filling the pores of the bone network. Old bone is normally broken down (i.e., resorbed) by cells called osteoclasts and replaced by bone-building cells called osteoblasts. This process of renewal is termed bone turnover. Osteoporosis occurs when the pores of the inner honeycomb or network become bigger by a predominance of bone resorption without concurrent restoration of new bone in the network, i.e., the bone becomes more porous, making the bone fragile and liable to break easily. Osteoporosis usually affects the whole skeleton, but it most commonly causes breaks (fractures) to bones in the wrist, spine, and hip. The elderly are at greatest risk of osteoporosis. The problem is therefore predicted to increase significantly with the aging of the population. Worldwide fracture incidence is predicted to increase three-fold over the next 60 years. In addition to the widespread occurrence of osteoporosis, a number of other metabolic bone diseases, such as osteopenia and Paget's Disease, are known that are also characterized by a loss of bone growth in an individual.

There are a number of causes of osteoporosis. Hormone deficiencies (estrogen in women, androgen in men) are the leading cause. It is well known that women are at greater risk of osteoporosis than men. Women experience a sharp acceleration of bone loss during the five years following menopause. Other factors that increase the risk of osteoporosis include smoking, alcohol abuse, a sedentary lifestyle, and low calcium intake.

Among the most common therapies currently employed for treating post-menopausal osteoporosis are hormone replacement therapy (HRT), bisphosphonates, and calcitonin. These three treatments work as anti-resorptive agents. Other adjuncts to these therapies may be recommended including adequate calcium intake, vitamin D supplements, and weight bearing exercise.

Estrogen is known to reduce fractures and is an example of an anti-resorptive agent. In addition, Black et al. (EP 0605193A1) report that estrogen, particularly when taken orally, lowers plasma levels of low density lipoproteins (LDLs), raises levels of the beneficial high density lipoproteins (HDLs), and prevents colorectal cancer. However, estrogen has failed to restore bone back to young adult levels in the established osteoporotic skeleton. Moreover, long-term estrogen therapy has been recently implicated in a variety of disorders, including an increase in the risk of breast cancer, stroke, and cardiovascular infarction, causing many women to avoid this treatment. The significant undesirable effects associated with estrogen replacement therapy support the need to develop alternative therapies for osteoporosis without undesirable side effects or health risks.

Bisphosphonates provide one form of non-hormonal treatment for osteoporosis that works by “switching off” the resorptive activity of osteoclasts and permitting osteoblasts to work more efficiently at producing new bone. There are several bisphosphonate compounds available on the market, including alendronate sodium (e.g., FOSAMAX®, Merck & Co., Inc., Whitehouse Station, N.J.), etidronate disodium and calcium carbonate (e.g., DIDRONEL PMO®, Procter & Gamble Co., Cincinnati, Ohio), and risedronate sodium (e.g., ACTONEL®, Aventis Pharmaceuticals, Parsippany, N.J.). Such compounds may provide a beneficial effect. For example, studies show that the risk of spinal fracture in post-menopausal women treated with the FOSAMAX® brand bisphosphonate are reduced by nearly 50% (see, e.g., Bone et al., N. Engl. J. Med., 350: 1189-1199 (2004)).

Calcium and vitamin D supplements are an effective treatment to reduce bone loss in the elderly. Most people can obtain adequate calcium in their diet, but supplements are an alternative for people who find this difficult. Calcium alone has a limited effect as a treatment for osteoporosis, but combined with vitamin D, it is particularly helpful for the elderly and housebound individual who cannot obtain natural sunlight and may have a poor diet.

Calcitriol is an active form of vitamin D given to post-menopausal women who have osteoporosis in the spine. Calcitriol improves the absorption of calcium from the gut, as calcium cannot be absorbed without vitamin D.

Calcitonin is a hormone made by the thyroid gland that prevents osteoclasts that break down bone from working properly and, thereby, improving the action of bone building osteoblasts. The drug acts by slowing the rate of bone loss and relieves bone pain. However, drawbacks with calcitonin are that it must be injected daily, it can cause nausea, and it is expensive compared with estrogen replacement therapy.

Testosterone is a treatment for men who are deficient in this male sex hormone, but it can also increase bone density in men with osteoporosis who have normal testosterone levels. It is available as injections or implants.

Anabolic steroids can increase bone and muscle mass and may be helpful in the very elderly who are frail and also in people with spinal fractures. Injections are carefully monitored due to side effects.

Selective estrogen receptor modulators (SERMs) are synthetic hormone replacement molecules that reduce the risk of osteoporosis and heart disease, but do not increase the risk of breast or endometrial cancers. One form, raloxifene, is approved for the prevention and treatment of osteoporosis in post-menopausal women.

Parathyroid hormone (PTH) has been approved for treating women with postmenopausal osteoporosis as the only available anabolic drug. Parathyroid hormone injected daily in small amounts can increase the formation of new bone, increase bone density, and decrease the likelihood of fractures.

For more than 30 years, bone morphogenetic proteins (BMPs, also called morphogens), a particular subclass of the transforming growth factor-β (TGF-β) super family of proteins, have been studied to understand the role these proteins play not only in bone and cartilage formation but also in soft tissue regeneration (e.g., kidney, heart, eye) and to develop such understanding into clinically effective therapies (see, e.g., Hoffmann et al., Appl. Microbiol. Biotechnol., 57: 294-308 (2001); Reddi, J. Bone Joint Surg., 83-A(Supp. 1): S1-S6 (2001); U.S. Pat. Nos. 4,968,590; 5,011,691; 5,674,844; 6,333,312). The use of a recombinant human BMP-7 in an osteogenic device that is surgically implanted into bone fractures to promote repair of non-union fractures has been reported (Friedlaender et al., J. Bone Joint Surg. Am. 83-A: S151-S158 (2001)).

For decades, the teaching in the field of BMPs has been that, unlike most proteins, BMPs are acid-stable and protease-stable and, thus, well-suited for use as orally administered therapeutic drugs that are not degraded by digestive enzymes and acids present in the mammalian digestive system (see, e.g., U.S. Pat. Nos. 4,968,590; 5,674,844; 6,333,312). Yet, despite issuance of U.S. patents describing use of BMPs for various therapeutic treatments, including methods for treating metabolic bone diseases (e.g., U.S. Pat. Nos. 5,674,844; 6,333,312), no clinical regimen comprising an oral formulation of a BMP to treat any metabolic disease appears to have been actually developed or approved. This may be because BMPs in fact are very sensitive to degradation by specific gastrointestinal enzymes, a fact that is demonstrated empirically herein for the first time.

Without advances in treating osteoporosis, all estimates of disease, fractures, and costs are expected to increase as the population of individuals over the age of 50 years old in the United States continues to increase for decades into the future.

Clearly, needs remain for effective treatments for osteoporosis and, indeed, for other metabolic bone diseases characterized by the loss of bone in an individual.

### SUMMARY OF THE INVENTION

The invention described herein solves the above problems for treating osteoporosis and other metabolic bone diseases by providing methods and compositions for the effective oral administration of a bone morphogenetic protein (BMP) to an individual. The invention is based on the discovery that, contrary to the historic and accepted teaching in the art, BMP molecules are sensitive to protease degradation by specific proteases present in the digestive system of humans and other mammals. Specifically, it has now been discovered that BMP molecules, such as BMP-6, are readily degraded in the mammalian stomach by the protease pepsin and in the intestines by the protease chymotrypsin. Orally (or “enterally”) administrable formulations of the invention encompass compositions that may be administered along the alimentary canal of an individual. Accordingly, formulations of the invention comprising a BMP that can be administered by way of the mouth of an individual must prevent degradation of the BMP in the stomach by gastric pepsin and also in the intestinal tract by intestinal chymotrypsin. Such formulations comprise an agent to prevent or inhibit proteolytic activity of gastric pepsin and also an agent to prevent or inhibit proteolytic activity of intestinal chymotrypsin. Formulations that are to be administered directly into the intestines, e.g., by injection or suppository, contain an agent to prevent or inhibit proteolytic activity of intestinal chymotrypsin, however, because the stomach is avoided, the presence of an agent to prevent or inhibit proteolytic activity of gastric pepsin is not required, i.e., is optional. The orally administrable formulations described herein permit an effective amount of BMP to be absorbed into the bloodstream of an individual to significantly restore and/or enhance bone growth, including bone mineral density, a parameter of bone growth that is critical for effectively treating osteoporosis and various other metabolic bone diseases. It is also appreciated that the oral formulations described herein may also find use in administering BMPs orally to an individual to treat a disease or disorder other than a metabolic bone disease.

In one embodiment, the invention provides a method of treating a metabolic bone disease that is characterized by a loss of bone mass in an individual comprising orally (enterally) administering to the individual a formulation comprising:
an osteoinductive bone morphogenetic protein (BMP) or functionally equivalent osteoinductive protein,
an agent to prevent or inhibit proteolytic activity of intestinal chymotrypsin, and
optionally, an agent to prevent or inhibit proteolytic activity of gastric pepsin.

Osteoinductive BMPs useful in the methods and formulations described herein include, without limitation, BMP-2, BMP-6, BMP-7, BMP-9, BMP-12, BMP-13, and combinations thereof.

Agents that are useful in the formulations and methods described herein to prevent or inhibit proteolytic activity of intestinal chymotrypsin include, without limitation, a pH lowering agent, a chymotrypsin-specific inhibitor, and combinations thereof.

A pH lowering agent may be any buffering agent that will effectively lower the pH in the intestine, preferably below pH 5, or at least the microenvironment around a BMP that passes into or is administered to the intestinal tract.

Agents that inhibit proteolytic activity of intestinal chymotrypsin from degrading a BMP according to the invention include, but are not limited to, chymostatin, Z-L-phe chloromethyl ketone, α2-antiplasmin, aprotinin (also called bovine pancreatic trypsin inhibitor or BPTI), 6-aminohexanoic acid, α1-antitrypsin, 4-(2-aminoethyl)benzene sulfonyl fluoride hydrochloride, bromoenol lactone, diisopropyl fluorophosphate, ecotoin, N-acetyl-eglin C, gabexate mesylate, leupeptin trifluoroacetate salt, N-p-tosyl-L-phenylalanine chloromethyl ketone, soybean trypsin-chymotrypsin inhibitor, and the like.

Agents that are useful in the formulations and methods described herein to prevent or inhibit proteolytic activity of gastric pepsin include, but are not limited to, pepsin inhibitors, enteric coatings, gastric pH regulating agents, and combinations thereof.

Pepsin inhibitors are compounds that bind pepsin and inhibit its proteolytic activity. Pepsin inhibitors useful in the methods and compositions described herein include, without limitation, pepstatin A, pepsinostreptin, phenylmethylsulfonyl fluoride, and the like.

Enteric coatings are made of one or more compounds that are formulated to provide a coating, film, or other protective solid encapsulation that is stable and resistant to dissolution or degradation by the low pH or enzymes of the gastric environment but that readily dissolves at higher pH (e.g., greater than 5) as exists in the intestines. In this way, enteric coatings useful in the invention effectively shield a coated therapeutic compound, such as a BMP, from degradation and/or denaturation in the stomach by gastric enzymes and acids, but, upon passage into the intestines, where the pH is significantly more alkaline (e.g., pH around 6 or higher), will dissolve and release the therapeutic compound for absorption into the bloodstream.

Gastric pH regulating agents useful in the invention raise the pH in the stomach or at least the microenvironment around a formulation comprising a BMP, or a functionally equivalent osteoinductive protein, present in the stomach to above the typical pH of 3 for a period of time sufficient to permit an effective amount of the BMP, or a functionally equivalent osteoinductive protein, to pass into the higher pH environment of the intestines without significant degradation by gastric pepsin. Gastric pH regulating agents useful in the invention may include, without limitation, buffering agents (“antacids”), histamine H2 receptor blockers (“H2 blockers”), and proton pump inhibitors.

In another embodiment, the methods and orally administrable formulations, as described herein, further comprise an inhibitor of trypsin, which can mediate a limited proteolytic degradation of a BMP in the duodenum and possibly other portions of the intestinal tract. As trypsin is active in the duodenum, agents analogous to those described above for inhibiting duodenal chymotrypsin may also be employed in such formulations, i.e., one or more pH lowering agents (buffers) and/or trypsin inhibitors.

In another embodiment, the methods and orally administrable formulations of the invention further comprise one or more agents to enhance the absorption of the osteoinductive BMP (or functionally equivalent osteoinductive protein) through the intestinal wall into the bloodstream. An absorption enhancer may be any of a variety of surface active agents or combinations of surface active agents. Preferred absorption enhancers useful in the invention include, but are not limited to, anionic agents that are cholesterol derivatives, cationic surface active agents, non-ionic surface active agents, and combinations thereof.

A variety of metabolic bone diseases that cause loss of bone growth may be treated with the methods and oral formulations described herein including, but not limited to, osteoporosis, osteopenia, osteomalacia, Paget's Disease, drug-induced (e.g., steroid-induced) osteopenia, drug-induced osteomalacia, nutritional rickets, metabolic bone disease associated with gastrointestinal disorders, metabolic bone disease associated with biliary disorders, tumor-associated bone loss, hypophosphatasia, and renal osteodystrophy.

### BRIEF DESCRIPTION OF THE DRAWINGS

FIG. 1 is a graph of results of a study using intravenous (i.v.) administration of BMP-6 in a rat model of osteoporosis described in Example 1. The graph shows selected bone mineral density (BMD) data for hind limbs of Sprague-Dawley female rats scanned before and after ovariectomy (OVX), except for Sham animals, over a 12-week course of treatment. Treatment Group 1 (Sham, no OVX, triangles), treatment Group 2 (OVX control, acetate buffer (vehicle) alone, i.v., 3 times/week, squares), and treatment Group 5 (OVX treated with 50 μg of BMP-6 per kg body weight (μg/kg), i.v., 3 times/week, diamonds). Vertical arrow indicates initiation of treatment (week 0) at 12 months (week-48) post OVX. Double asterisks indicate statistical significance (P<0.005) for differences between data point of Group 5 (OVX treated with BMP-6, diamonds) compared to the corresponding point of Group 2 (OVX control, acetate buffer, squares). See text for details.

FIG. 2 is a bar graph that shows BMD values in hind limbs of animals of a rat model of osteoporosis in all treatment groups of the study described in Example 1 at twelve weeks from commencement of treatment. Group 1 (Sham), Group 2 (OVX control, acetate buffer alone, 3 times/week), Group 3 (OVX treated with BMP-6, 10 μg/kg, i.v., 3 times/week), Group 4 (OVX treated with BMP-6, 25 μg/kg, i.v., 3 times/week), Group 5 (OVX treated with BMP-6, 50 μg/kg, i.v., 3 times/week), Group 6 (OVX treated with estradiol, 175 μg/week, administered to each animal as 3 separate doses: 50 μg, 50 μg. and 75 μg/rat, subcutaneously, s.c.), Group 7 (OVX treated with estradiol+BMP-6; estradiol: 175 μg/week, administered to each animal as 3 separate doses: 50 μg, 50 μg, and 75 μg/rat, s.c.; BMP-6: 10 μg/kg, i.v., 3 times/week). The difference in BMD values of any of treatment Groups 3, 4, 5, or 7 and that of Group 2 (OVX control, acetate buffer alone) is significant (P<0.001), as is the difference in BMD values of Groups 3, 4, 5, or 7 and that of treatment Group 6 (OVX treated with estradiol alone) (P<0.004). See text for details.

FIG. 3 is a bar graph that shows BMD values for lumbar spine of animals in the study in Example 1 at twelve weeks from commencement of treatment. The difference in BMD values of any of treatment Groups 3, 4, 5, or 7 and that of Group 2 (OVX control, acetate buffer alone) or that of Group 6 (OVX treated with estradiol alone) is significant (P<0.001). See text for details.

FIG. 4 is a bar graph of results of the study in Example 1, wherein BMD values were determined by ex vivo DEXA analysis of distal femurs of animals after sacrifice. Differences between the BMD value of treatment Groups 1 (Sham), 3, 4, 5, 6, or 7 and Group 2 (OVX control, acetate buffer alone) were statistically significant (P<0.001). BMD values of treatment Groups 3, 4, 5, and 7 are much higher than the BMD value of Group 6 (OVX treated with estradiol alone). See text for details.

FIG. 5 is a bar graph of bone mineral area (BMA) in distal femurs of animals of selected treatment Groups in the study described in Example 1. Cortical BMA values were 25% higher in BMP-6 treated Groups 3, 5, and 7 as compared to that of animals of treatment Group 2 (OVX control, acetate buffer alone). See text for details.

FIG. 6 is a bar graph for the ratio of bone volume/trabecular volume (BV/TV) for distal femurs of animals in treatment Groups 1 (Sham), 2 (OVX control, acetate buffer alone), 3 (OVX treated with BMP-6, 10 μg/kg), 6 (OVX treated with estradiol), and 7 (OVX treated with estradiol +BMP-6) as described in Example 1. See text for details.

FIG. 7 is a bar graph for trabecular bone thickness (mm) for animals in treatment Groups 1 (Sham), 2 (OVX control, acetate buffer alone), 3 (OVX treated with BMP-6, 10 μg/kg), 6 (OVX treated with estradiol alone), and 7 (OVX treated with estradiol+BMP-6) as described in Example 1. See text for details.

FIG. 8 is a bar graph for indentation test expressed as maximal load (Force, in newtons, “N”) of cancellous bone in the marrow cavity of the distal femoral metaphysis (DFM) of animals of treatment Groups 1 (Sham), 2 (OVX control, acetate buffer alone), and 3 (OVX treated with BMP-6, 10 μg/kg, i.v.) of the study described in Example 1. Asterisk indicates statistical significance (P<0.001) for difference between Group 3 and Group 2. See text for details.

FIG. 9 is a bar graph for the absorbed energy parameter of a three-point bending test of midshaft femur expressed as Work (W, millijoules, “mJ”) of animals of treatment Groups 1 (Sham), 2 (OVX control, acetate buffer alone), and 3 (OVX treated with BMP-6, 10 μg/kg, i.v.) of the study described in Example 1. See text for details.

FIG. 10 is a bar graph for the toughness (a derived parameter) of a three-point bending test of midshaft femur expressed as millijoules/m³ (mJ/m³) of animals of treatment Groups 1 (Sham), 2 (OVX control, acetate buffer alone), and 3 (OVX treated with BMP-6, 10 μg/kg, i.v.) of the study described in Example 1. See text for details.

FIG. 11 is a bar graph of the ratio of bone volume to trabecular bone volume (BV/TV) based on histomorphometric analysis of distal femurs of animals of treatment Groups 1 (Sham), 2 (OVX control, acetate buffer alone), 3 (OVX treated with BMP-6, 10 μg/kg, i.v.), and 6 (OVX treated with estradiol alone) of the study described in Example 1. See text for details.

FIG. 12 is a bar graph of BV/TV values based on dynamic histomorphometric analysis to measure mineral apposition rate (“MAR”, μm/day) of distal femurs of animals of treatment Groups 1 (Sham), 2 (OVX control, acetate buffer alone), 3 (OVX treated with BMP-6, 10 μg/kg, i.v.), and 6 (OVX treated with estradiol alone) of the study described in Example 1. Asterisks indicate statistical significance (P<0.001) for BMP-treated animals (Group 3) and animals treated with estradiol+BMP-6 (Group 6) compared to ovariectomized control animals (Group 2). See text for details.

FIG. 13 is a bar graph of BMD values for hind limbs of aged (2 years, 1 month old), ovariectomized (OVX) rats as described in Example 2 for treatment Groups 1 (Sham, no OVX), 2 (OVX control, acetate buffer alone), 3 (OVX treated with BMP-6, 10 μg/kg, i.v., 3 times/week), 4 (OVX treated with BMP-6, 10 μg/kg, i.v., 1 time/week), 5 (OVX treated with BMP-6, 1 μg/kg, i.v., 3 times/week). See text for details.

FIG. 14 is a graph of BMD values of hind limbs of animals as a function of time of treatment (weeks) in the study described in Example 2 for treatment Groups 1 (Sham, triangles), 2 (OVX control, acetate buffer alone, squares), and 5 (OVX treated with BMP-6, 1 μg/kg, i.v., 3 times/week, diamonds). Arrow indicates initiation of treatment (week 0). See text for details.

FIG. 15 shows a graph of the percentage of orally administered BMP-6 that was absorbed in rats as a function of age and route (i.e., via mouth) as described in Example 3. Animals received 99m technetium-labeled BMP-6 administered orally by mouth (3 days old in Group 1; 15 days old in Group 2) or duodenally by syringe (45 days old in Group 3, 75 days old in Group 4). See text for details.

FIG. 16 is a bar graph of the percentage of duodenally administered BMP-6 absorbed in Animal 1 (BMP-6 in acetate buffer, pH 3), Animal 2 (BMP-6, acetate buffer, pH 3, taurodeoxycholic acid sodium (1 mg) and DL-lauroylcarnitine chloride (1 mg)), and Animal 3 (BMP-6, 0.9% NaCl, pH 7, taurodeoxycholic acid sodium (1 mg) and DL-lauroylcarnitine chloride (1 mg)) as described in Example 3. See text for details.

FIG. 17 is a bar graph showing the absorption of intraduodenally (i.d.) administered BMP-6 expressed as percentage of intravenous dose for animals in treatment Groups 1 (BMP-6, i.d, acetate buffer, pH 4), 2 (BMP-6, i.d., acetate buffer, pH 3, 1 mg taurodeoxycholic acid, 1 mg DL-lauroyl carnitine chloride), 3 (BMP-6, i.d., acetate buffer, pH 3, 1 mg taurodeoxycholic acid, 1 mg DL-lauroyl carnitine chloride, 1.5 mg diheptanoylphosphatidylcholine), 4 (BMP-6, i.d., acetate buffer, pH 3, 1.5 mg diheptanoylphosphatidylcholine), and 5 (BMP-6, i.v., acetate buffer, pH 4) as described in Example 5. See text for details.

FIGS. 18A and 18B show bar graphs of results (in millions of counts per minute) of a study as described in Example 6 of transference of 99mTc-labeled BMP-6 from mucosal (M, external) to serosal (S, internal) surface in an everted gut system incubated for 0 and 90 minutes in a non-buffering incubation Medium 1 (FIG. 18A) or a buffered (pH 7.4) incubation Medium 2 (FIG. 18B). See text for details.

FIG. 19 is a polyacrylamide gel showing the digestion products reduced with dithiotbreitol to release BMP-6 monomer, electrophoresed, and stained with Coomassie Blue, from various reactions: BMP-6 incubated in the presence of 0, 10, 5, and 1 μL of pepsin (lanes 1-4, respectively); BMP-6 and bovine serum albumin (BSA) incubated in the presence of 5 and 1 μL of pepsin (lanes 6 and 7, respectively); and BSA incubated in the presence of 5 and 1 μL of pepsin (lanes 8 and 9, respectively), as described in Example 7. Lane 5 contains molecular weight markers. The relative positions of BSA, pepsin, and the BMP-6 monomer in the gel are indicated by horizontal arrows. See text for details.

FIG. 20 is a polyacrylamide gel showing the digestion products reduced with dithiothreitol to release BMP-6 monomer, electrophoresed, and stained with Coomassie Blue, from various reactions: BMP-6 incubated in the presence of 0, 1, and 0.2 μL trypsin (lanes 1, 2, 3, respectively); BMP-6 incubated in the presence of 0.5 and 0.2 μL chymotrypsin (lanes 5 and 6); BMP-6 and BSA incubated in the presence of 0.2 μL trypsin (lane 7); BMP-6 and BSA incubated in the presence of 0.2 μL chymotrypsin (lane 8); BSA incubated in the presence of 0.2 μL trypsin (lane 9); and BSA incubated in the presence of 0.2 μL chymotrypsin (lane 10), as described in Example 7. Lane 4 contains molecular weight markers. The relative positions of BSA and the BMP-6 monomer in the gel are indicated by horizontal arrows. See text for details.

FIG. 21 is a Western immunoblot of a polyacrylamide gel showing the digestion products, electrophoresed and immunodetected (stained), from various reactions as described in Example 7. Dithiothreitol was added to reaction mixture prior to electrophoresis to detect BMP-6 monomer or withheld (no DTT) to detect BMP-6 dimer. BMP-6 incubated in the presence of 10, 1, and 1 μL of gastric juice from animal 1 (lanes 2, 3, 4 (no DTT), respectively); BMP-6 incubated in the presence of 10, 1, and 1 μl of gastric juice from animal 2 (lanes 5, 6, 7 (no DTT), respectively); BMP-6 incubated in the presence of 10, 1, and 1 μL of heat-inactivated gastric juice (lanes 8, 9, and 10 (no DTT), respectively). Molecular weight markers were run in lane 1 and also in lane 10. See text for details. The relative positions of pepsin in the stomach juice, the BMP-6 dimer, a partially digested “damaged BMP-6 dimer”, and the BMP-6 monomer are indicated by horizontal arrows. See text for details.

FIG. 22 is a Western immunoblot of a polyacrylamide gel showing the digestion products, electrophoresed and immunodetected (stained), from various reactions as described in Example 7. Dithiothreitol was added to reaction mixture prior to electrophoresis to detect BMP-6 monomer or withheld (no DTT) to detect BMP-6 dimer. BMP-6 incubated in the presence of 10, 1, and 1 μL of gastric juice from animal 1 (lanes 1, 2, and 3 (no DTT), respectively) and from animal 2 (lanes 4, 5, and 6 (no DTT), respectively); BMP-6 incubated in the presence of the pepsin inhibitor pepsinostreptin and 10, 1, and 1 μL of gastric juice (lanes 7, 8, and 9 (no DTT), respectively); and BMP-6 incubated in the presence of 10, 1, and 1 μL of heat-inactivated gastric juice (lanes 11, 12, and 13 (no DTT), respectively). BMP-6 monomer was run in lane 14. BMP-6 dimer (no DTT) was run in lane 15. Molecular weight markers were run in lane 10. The relative positions of the BMP-6 dimer and the BMP-6 monomer are indicated by horizontal arrows. See text for details.

FIG. 23 is a Western immunoblot of a polyacrylamide gel showing the digestion products, electrophoresed and immunodetected (stained), from various reactions as described in Example 7. Dithiothreitol was added to reaction mixture prior to electrophoresis to detect BMP-6 monomer or withheld (no DTT) to detect BMP-6 dimer. BMP-6 incubated in the presence of 3 and 1 μL of duodenal juice from animal 1 (lanes 1 and 2, respectively) and from animal 2 (lanes 3 and 4, respectively); BMP-6 incubated in the presence acetate buffer (pH 3) and 3, 1, and 1 μL of duodenal juice (lanes 6, 7, and 8 (no DTT), respectively); and BMP-6 incubated in the presence of 1 μL of heat-inactivated duodenal juice (lane 9). BMP-6 monomer was run in lane 10. BMP-6 dimer (no DTT) was run in lane 11. Molecular weight markers were run in lane 5. The relative positions of the BMP-6 dimer, the BMP-6 monomer, and a “truncated BMP-6” are indicated by horizontal arrows. See text for details.

FIG. 24 is a Western immunoblot of a polyacrylamide gel showing digestion products, electrophoresed and immunodetected (stained), from various reaction mixtures as described in Example 7. Dithiothreitol was added to reaction mixture prior to electrophoresis to detect BMP-6 monomer (lanes 8, 9, 10, and 11) or withheld (no DTT) to detect BMP-6 dimer (lanes 2, 3, 4, and 5). BMP-6 incubated in the presence of 1 μl of duodenal juice (lanes 2 and 8); BMP-6 incubated in the presence of 1 μL of duodenal juice and 1 μL of the chymotrypsin inhibitor chymostatin (lanes 3 and 9); BMP-6 incubated in the presence of 1 μL of duodenal juice and 1 μL soybean trypsin inhibitor (lanes 4 and 10); and BMP-6 incubated in the presence of 1 μL of duodenal juice and 1 μL aprotinin (lanes 5 and 11). BMP-6 dimer (no DTT) was run in lane 1. BMP-6 monomer was run in lane 7. Molecular weight markers were run in lane 6. The relative positions of the BMP-6 dimer and the BMP-6 monomer are indicated by horizontal arrows. See text for details.

FIG. 25 is a Western immunoblot of a polyacrylamide gel showing digestion products, electrophoresed and immunodetected (stained), from various reaction mixtures as described in Example 7. Dithiothreitol was added to reaction mixture prior to electrophoresis to detect BMP-6 monomer (lanes 2, 3, 4, and 5) or withheld (no DTT) to detect BMP-6 dimer (lanes 6, 7, 8, and 9). BMP-6 incubated in the presence of 1 μL of duodenal juice and pH 7 buffer (lanes 3 and 7); BMP-6 incubated in the presence of 1 μL of duodenal juice and pH 4 buffer (lanes 4 and 8); and BMP-6 incubated in the presence of 1 μL of duodenal juice and pH 5 buffer (lanes 5 and 9). BMP-6 monomer was run in lane 2. BMP-6 dimer (no DTT) was run in lane 6. The relative positions of the BMP-6 monomer and the BMP-6 dimer are indicated by horizontal arrows. See text for details.

FIG. 26 is a graph of results of a study using enteral administrations of BMP-6 in a rat model of osteoporosis as described in Example 8. The graph shows selected bone mineral density (BMD) data for hind limbs of Sprague-Dawley female rats scanned before and after ovariectomy (OVX), except for Sham animals. Treatment Group 1 (Sham, no OVX, n=15, black diamonds), treatment Group 2 (OVX control, acetate buffer alone, pH 3.5, intraduodenally, i.d., n=10, small squares), treatment Group 3 (OVX treated with 500 μg/kg BMP-6, 20 μg chymotrypsin, 20 μg aprotinin, pH 7.0, i.d., once per week, n=14, triangles), treatment Group 4 (OVX treated with 500 μg/kg BMP-6, 20 μg chymotrypsin, 20 μg aprotinin, pH 3.5, i.d., once per week, n=14, large squares), and treatment Group 5 (OVX treated with 300 μg/kg BMP-6, 50 μg chymotrypsin, 50 μg aprotinin, 50 μg pepstatin, pH 3.5, per os delivery with gastric tube, three times per week, n=14, white diamonds). Vertical arrow indicates initiation of treatment at 6 months post OVX (time 0). Double asterisks indicate statistical significance (P<0.005) for difference between data point of Group 3 (triangle) compared to the corresponding point of Group 2 (OVX control, acetate buffer, small square). Single asterisk indicates statistical significance (P<0.05) for difference between data point of Group 4 (large square) and corresponding data point of control Group 2 (small square). See text for details.

FIG. 27 is a bar graph that shows BMD values in hind limbs of animals of rat model of osteoporosis at 3 weeks after receiving treatments for a 3-week period for treatment Groups of the study described above for FIG. 26 (Example 8). Treatment Group 1 (Sham), Group 2 (OVX control, acetate buffer alone, pH 3.5, intraduodenally, i.d.), Group 3 (500 μg/kg body weight BMP-6, 20 μg chymotrypsin, 20 μg aprotinin, pH 7.0, i.d., once per week), and treatment Group 4 (500 μg/kg BMP-6, 20 μg chymotrypsin, 20 μg aprotinin, pH 3.5, i.d., once per week). Double asterisks indicate statistical significance (P<0.005) for difference between BMD of treatment Group 3 compared to the BMD of Group 2 (OVX control, acetate buffer, squares). Single asterisk indicates statistical significance (P<0.05) for the differences between BMD of treatment Group 4 or Group 5 and the BMD of control Group 2. See text for details.

### DETAILED DESCRIPTION OF THE INVENTION

The invention provides compositions comprising a bone morphogenetic protein (BMP), or a functionally equivalent osteoinductive protein, for use as an orally administered treatment for osteoporosis and other metabolic bone diseases that are characterized by loss of bone growth or mass in an individual. Such oral formulations of BMPs may comprise one or more agents that prevent or inhibit proteolytic activity of gastric pepsin and of intestinal chymotrypsin such that an effective amount of a BMP may pass from the stomach into the intestinal tract and ultimately be absorbed into the bloodstream of an individual.

In order that the invention may be more clearly understood, the following terms are used as defined below.

“Bone morphogenetic protein”, “BMP”, and “morphogen” are synonymous and refer to any member of a particular subclass of the transforming growth factor-β (TGF-β) super family of proteins (see, e.g., Hoffmann et al., Appl. Microbiol. Biotechnol., 57: 294-308 (2001); Reddi, J. Bone Joint Surg., 83-A(Supp. 1): S1-S6 (2001); U.S. Pat. Nos. 4,968,590; 5,011,691; 5,674,844; 6,333,312). All BMPs have a signal peptide, prodomain, and a carboxy-terminal (mature) domain. The carboxy-terminal domain is the mature form of the BMP monomer and contains a highly conserved region characterized by seven cysteines that form a cysteine knot (see, Griffith et al., Proc. Natl. Acad. Sci. USA., 93: 878-883 (1996)).

BMPs were originally isolated from mammalian bone using protein purification methods (see, e.g., Urist et al., Proc. Soc. Exp. Biol. Med., 173: 194-199 (1983); Urist et al., Proc. Natl. Acad. Sci. USA, 81: 371-375 (1984); Sampath et al., Proc. Natl. Acad. Sci. USA, 84: 7109-7113 (1987); U.S. Pat. No. 5,496,552). However, BMPs have also been detected in or isolated from other mammalian tissues and organ including kidney, liver, lung, brain, muscle, teeth, and gut. BMPs may also be produced using standard in vitro recombinant DNA technology for expression in prokaryotic or eukaryotic cell cultures (see, e.g., Wang et al., Proc. Natl. Acad. Sci. USA, 87: 2220-2224 (1990); Wozney et al., Science, 242: 1528-1534 (1988)). Some BMPs are commercially available for local use as well (e.g., BMP-7 is manufactured and distributed for treatment of long bone non-union fractures by Stryker-Biotech (Hopkinton, Mass., U.S.); BMP-2 is manufactured and distributed for long bone acute fractures by Wyeth (Madison, N.J., U.S.), and also for spinal fusions by Medtronic, Inc., Minneapolis, Minn., U.S.).

BMPs normally exist as dimers of the same monomeric polypeptides (homodimers) held together by hydrophobic interactions and at least one interchain (between monomers) disulfide bond. However, BMPs may also form heterodimers by combining the monomers of different degrees (lengths) of processing (e.g., a full-length, unprocessed monomer associated with a processed, mature monomer) or monomers from different BMPs (e.g., a BMP-6 monomer associated with a BMP-7 monomer). A BMP dimer of unprocessed monomers or a BMP heterodimer of one processed BMP monomer and one unprocessed BMP monomer are typically soluble in aqueous solutions, whereas a BMP homodimer comprised of two fully processed (mature) monomers is only soluble in an aqueous solution at a low pH (e.g., acetate buffer, pH 4.5) (see, e.g., Jones et al., Growth Factors, 11: 215-225 (1994)).

BMPs useful in the invention are those that have osteoinductive activity, i.e., the ability to stimulate bone formation. Osteoinductive (or “osteogenic”) activity may be detected using any of a variety of standard assays. Such osteoinductive assays include ectopic bone formation assays in which a carrier matrix comprising collagen and a BMP are implanted at an ectopic site in a rodent, and the implant then monitored for bone formation (Sampath and Reddi, Proc. Natl. Acad. Sci. USA, 78: 7599-7603 (1981)). In a variation of such an assay, the matrix may be implanted at an ectopic site and the BMP administered to the site, e.g., by intravenous injection into the rodent (see, also Examples 4 and 9, below). Another way to assay for BMP osteoinductive activity is to incubate cultured fibroblast progenitor cells with a BMP and then monitor the cells for differentiation into chondrocytes and/or osteoblasts (see, e.g., Asahina et al., Exp. Cell. Res. 222: 38-47 (1996)). BMPs that have osteoinductive activity and that are therefore useful in the invention include, but are not limited to, BMP-6, BMP-2, BMP-4, BMP-7, BMP-9, BMP-12, BMP-13, and heterodimers thereof, whether purified from a natural source, produced recombinantly, or produced in whole or in part by in vitro protein synthesis methods. A BMP that has an osteoinductive activity may also possess one or more other beneficial pharmacological activities such as the ability to restore or regenerate damaged soft tissues or organs, e.g., ischemic kidneys (Vukicevic et al., J. Clin. Invest., 102: 202-214 (1998)).

It is also understood that compositions and methods comprising a BMP as described herein may alternatively comprise a protein other than a known osteoinductive BMP provided such protein is functionally equivalent to a BMP in that the protein has an osteoinductive activity as indicated by a standard osteoinductive assay such as those described above (e.g., a fibroblast progenitor to chondrocyte/osteoblast differentiation assay). For the purposes of the present invention, it is presumed that such alternative osteoinductive proteins will exhibit a like sensitivity to gastrointestinal degradation, and thus will benefit from the presence of protective agents in making an oral formulation according to the invention. However, to the extent that such osteoinductive proteins are not as susceptible as BMPs to such enzymatic degradation, decreasing or eliminating enzyme-neutralizing agents to make an effective oral formulation may be possible. Functionally equivalent proteins may include various BMP homologues, i.e., proteins that have an amino acid sequence that is homologous to a known osteoinductive BMP and that are susceptible to degradation by pepsin and chymotrypsin. Such BMP homologues may be naturally occurring, recombinantly produced, or synthetically produced in whole or in part (see, e.g., U.S. Pat. Nos. 5,674,844; 6,333,312).

Unless stated otherwise, the terms “disorder” and “disease” are synonymous, and refer to any pathological condition irrespective of cause or etiological agent.

A “drug” refers to any compound (e.g., a protein, peptide, organic molecule) or composition that has a pharmacological activity. Thus, a “therapeutic drug” is a compound or composition that can be administered to an individual to provide a desired pharmacological activity to treat a disease, including amelioration of one or more symptoms of a disease. A “prophylactic drug” is a compound or composition that can be administered to an individual to prevent or provide protection from the development in an individual of a disease. A drug may have prophylactic as well as therapeutic uses. For example, treating an individual for osteoporosis or other metabolic bone disease with an orally administered composition according to the invention promotes healthy bone growth, which in turn protects the individual from developing a heightened susceptibility to bone fractures, skeletal deformation, and other complications associated with advanced stages of osteoporosis and other metabolic bone diseases. Accordingly, unless indicated otherwise, a “treatment” of (or “to treat”) a disease according to the invention comprises enteral administration of a formulation described herein to an individual to provide therapeutic and/or prophylactic benefits to the individual.

The terms “composition”, “formulation”, “preparation”, and the like are synonymous and refer to a composition that may consist of one or more compounds. Oral formulations comprising a BMP as described herein for treating osteoporosis or other metabolic bone diseases are specifically formulated to prevent or inhibit proteolytic degradation of the BMP by gastric pepsin and/or duodenal chymotrypsin. “Metabolic bone disease (or disorder)” refers to any pathology of bone growth that is not directly the result of physical trauma. Metabolic bone diseases include, but are not limited to, osteoporosis, osteopenia, Paget's Disease (osteitis deformans), and osteomalacia. Other bone disorders that may be treated by this invention include, but are not limited to, drug-induced (e.g., steroid-induced) osteopenia, nutritional rickets, metabolic bone disease associated with gastrointestinal disorders, metabolic bone disease associated with biliary disorders, tumor-associated bone loss, hypophosphatasia, drug-induced osteomalacia, and renal osteodystrophy.

“Osteoporosis” has the meaning known in medicine and the field of metabolic bone disease. As noted above, osteoporosis is a systemic skeletal disease that is characterized by low bone mass and deterioration of bone tissue with a consequent increase in bone fragility and susceptibility to fracture. Osteoporosis occurs when the pores of the inner honeycomb or network of normal bone become larger by a predominance of bone resorption without concurrent restoration of new bone in the network thereby making the bone fragile and liable to break easily. Osteoporosis usually affects the whole skeleton, but it most commonly causes breaks (fractures) to bones in the wrist, spine, and hip.

By “pharmaceutically acceptable” is meant a material that is not biologically, chemically, or in any other way, incompatible with body chemistry and metabolism and also does not adversely affect the desired, effective activity of a bone morphogenetic protein or any other component in a composition that may be administered to an individual to treat or prevent a disorder (e.g, osteoporosis or other metabolic disease) according to the invention.

A formulation described herein may be referred to as “oral”, “orally administrable”, “enteral”, “enterally administrable”, “non-parenteral”, “non-parenterally administrable”, and the like to indicate the route or mode for administering the formulation to provide an effective amount of a BMP to an individual anywhere along the alimentary canal. Examples of such “oral” or “enteral” routes of administration include, without, limitation, by the mouth, e.g., swallowing a solid (e.g., pill, tablet, capsule) or liquid (e.g., syrup) compound or composition; sub-lingual (absorption under the tongue); nasojejunal or gastrostomy tubes (into the stomach); intraduodenal (i.d.) administration (e.g., by individual injections or via a pump); and rectal administration (e.g., suppositories for administering a compound or composition into the lower intestinal tract for absorption). One or more oral (enteral) routes of administration may be employed in the invention. A particularly preferred route for administering a BMP to treat a metabolic bone disorder in an individual is to have the individual swallow a formulation described herein comprising a BMP and agents that prevent or inhibit gastric pepsin and duodenal chymotrypsin proteolytic activities. Thus, unless a particular type of “oral” formulation described herein is specified or otherwise indicated by the context or by a description of its particular ingredients, “oral” formulations are the same as “enteral” formulations and broadly encompass formulations that may be administered to an individual at one or more points along the alimentary canal.

Terms such as “parenteral” and “parenterally” refer to routes or modes of administration of a compound or composition to an individual other than along the alimentary canal. Examples of parenteral routes of administration include, without limitation, subcutaneous (s.c.), intravenous (i.v.), intramuscular (i.m.), intra-arterial (i.a.), intraperitoneal (i.p.), transdermal (absorption through the skin or dermal layer), nasal or pulmonary (e.g., via inhalation or nebulization, for absorption through the respiratory mucosa or lungs), direct injections or infusions into body cavities or organs, as well as by implantation of any of a variety of devices into the body that permit active or passive release of a compound or composition into the body.

Amino acid residues may be designated by full name or by the corresponding standard three-letter or one-letter abbreviations known in the art.

The meaning of other terms will be evident by the context of use and, unless otherwise indicated, are consistent with the meanings understood by those skilled in the fields of medicine, metabolic bone disorders, and pharmacology.

Despite over three decades of research, volumes of literature, and the issuance of U.S. patents purporting the potential use of BMPs for various therapeutic treatments, including methods for treating osteoporosis (see, e.g., U.S. Pat. Nos. 5,674,844; 6,333,312), no effective oral formulation or clinical regimen comprising oral administration of a BMP to treat a metabolic bone disease is available. As shown conclusively herein, the prior accepted teaching in the art that BMPs are resistant to degradation by digestive enzymes and acids in the mammalian digestive system and, therefore, readily amenable to oral formulations and therapies (Id.) is clearly incorrect. In particular, it has now been discovered that in order for an effective amount of an orally administered BMP to be absorbed into the body to produce an effective therapeutic result, the BMP must be protected from specific proteolytic activities of the gut, in particular, pepsin in the stomach and chymotrypsin in the duodenum (e.g., see, below, Examples 7 and 8). The elucidation of the specific proteolytic susceptibilities of BMPs now provides the basis for new and useful therapeutic oral formulations of these compounds to treat metabolic diseases and damaged tissues. As described with particularity herein, oral formulations comprising BMPs are useful to treat metabolic diseases such as osteoporosis and other metabolic bone diseases.

Gastric pepsin is proteolytically active in the acidic (pH 3) environment of the stomach. Chymotrypsin is active at higher pH ranges (e.g., pH 7), as generally found in the duodenum and intestinal tract. As explained in greater detail, below, oral formulations of the invention comprise a BMP (or a functionally equivalent osteoinductive protein) and one or more agents that prevent gastric pepsin and/or intestinal chymotrypsin access to (contact with) the BMP (or functionally equivalent osteoinductive protein) and/or that inhibit the proteolytic activities of these enzymes in the mammalian digestive tract (gut) and, thereby, permit an effective amount of the orally administered BMP (or functionally equivalent osteoinductive protein) to pass through the stomach and into the intestines for absorption into the bloodstream.

Agents for Preventing Degradation of BMP by Gastric Pepsin

Pepsin is a gastric enzyme that is active at pH 3 (as in the stomach) and irreversibly inactivated at a pH above 6. Pepsin preferentially cleaves a susceptible protein, polypeptide, or peptide at the carboxyl side of a phenylalanine (Phe), leucine (Leu), or glutamate (Glu) residue in the amino acid sequence of the protein, polypeptide, or peptide. The enzyme does not cleave bonds containing valine (Val), alanine (Ala), or glycine (Gly). Compositions for oral administration of a BMP according to the invention may comprise one or more agents that prevent gastric pepsin from degrading a BMP while in the stomach.

Formulations of the invention for oral administration of a BMP may be encased or otherwise sequestered from gastric enzymes and acids using any of a variety of enteric coatings. Such enteric coatings typically provide a coating, film, or other protective solid encapsulation that is stable and resistant to dissolution or degradation by the low pH or enzymes of the gastric environment but that readily dissolves at higher pH (e.g., greater than 5) as exists in the intestines. In this way enteric coatings useful in the invention shield an effective amount of BMP from degradation in the stomach by pepsin or any other gastric enzyme, and upon passage into the intestines, where the pH is significantly higher, will dissolve and release the BMP for absorption into the bloodstream. Enteric coatings useful in preparing BMPs for oral administration according to the invention may comprise any of a variety of pharmaceutically acceptable compounds that have the properties necessary to protect an orally delivered therapeutic agent from degradation or denaturation by the enzymes and/or acids of the stomach. Various pharmaceutically acceptable compounds are known for preparing such enteric coatings including, without limitation, cellulose acetate phthlate (“CAP”), cellulose acetate trimellite, hydroxypropylmethylcellulose phthlate, hydroxpropylmethyl cellulose acetate succinate, polyvinyl acetate phthlate, methacrylic acid copolymers, ethyl acrylate copolymers, and combinations thereof. Enteric coated formulations may be further encapsulated in various types of pharmaceutically acceptable, dissolvable shells.

A BMP may also be protected from degradation by gastric pepsin using a gastric pH regulating agent that raises the pH in the stomach or at least the microenvironment around the BMP present in the stomach to a level that is beyond the pH optimum for significant proteolytic activity by pepsin and for a period of time sufficient to permit the BMP to pass out of the stomach and into the intestinal tract. Pepsin-mediated protein degradative activity is noticeably lower at pH 4 and essentially inactivated at pH above 5. Accordingly, a gastric pH regulating agent useful in the formulations described herein may be any of a variety of buffering agents, also referred to as stomach “antacids”, that temporally raise the gastric pH in the range of from 4 to 7. Preferably, the gastric pH regulating agent raises the gastric pH to at least 5. Antacids that may be used in formulations described herein as gastric pH regulating agents include, without limitation, calcium carbonate, sodium bicarbonate, aluminum hydroxide, magnesium hydroxide, aluminum carbonate gel, and the like. Compounds that block histamine H2 receptors (“H2 blockers”) may also be used as gastric pH regulating agents in compositions and methods described herein. Such H2 blockers include, but are not limited to, cimetidine, famotidine, nizatidine, ranitidine, and the like. Yet another type of compound that may serve as a gastric pH regulating agent in the methods and compositions described herein are compounds that inhibit proton pumps. Such proton pump inhibitors include, but are not limited to, lansoprazole, omeprazole, pantoprazole, abeprazole, and the like. An appropriate amount of a gastric pH regulating agent to use in a formulation described herein is readily determined following the practices of those skilled in the art for preparing stomach antacids, H2 blockers, or proton pump inhibitors. Oral formulations of BMPs may also comprise more than one gastric pH regulating agent.

Gastric pepsin-mediated proteolysis of a BMP may also be prevented using one or more pepsin-specific inhibitor compounds that bind to pepsin and inhibit the proteolytic activity of the enzyme. Such pepsin inhibitors may include, but are not limited to, pepstatin A, pepsinostreptin, phenylmethylsulfonyl fluoride, and the like.

The effectiveness of a pepsin inhibitor, a pH regulating agent, or a combination thereof, to inhibit the proteolytic activity pepsin may be initially tested in a standard in vitro assay for pepsin-mediated proteolytic activity (see, e.g., Examples 7 and 8, below).

Agents for Preventing Degradation of BMP by Chymotrypsin

Chymotrypsin is a serine protease that hydrolyzes a peptide bond with aromatic or large hydrophobic side chains (as in amino acids Tyr, Trp, Phe, Met) on the carboxyl side of the peptide bond. Chymotrypsin is an intestinal enzyme that has an optimal pH of 7.8 for its proteolytic activity. A composition for oral administration of a BMP according to the invention may comprise one or more agents that prevent or inhibit the proteolytic activity of intestinal chymotrypsin so that an effective amount of the BMP present in the intestinal tract may be absorbed into the bloodstream.

The proteolytic activity of intestinal chymotrypsin may be inhibited by lowering the pH in the intestine or of at least the microenvironment around a BMP present in the intestine, e.g., in the duodenum, to a point where no significant proteolytic activity occurs during the time that the BMP is being absorbed into the bloodstream. Any of a variety of pharmaceutically acceptable pH lowering agents (buffer, buffering agent) may be used to effect a lowering of the pH, preferably below pH 5, and at least in the duodenum of the intestinal tract, e.g., when a formulation according to the invention passes into the duodenum from the stomach or is injected intraduodenally or rectally. An oral formulation of BMP according to the invention that is swallowed preferably releases a pH lowering agent only after the BMP has passed into the duodenum. Buffers useful for lowering the pH of at least the microenvironment of BMP that has passed into the duodenum include, but are not limited to, acetate, succinate, lactate, citrate, isocitrate, ascorbate, oxaloacetate, oxalate, malate, fumarate, 2-ketoglutarate, glutarate, pyruvate, glycerate, and combinations thereof. It is understood that referring to a buffer by the salt form of an acid also encompasses the corresponding acid form as may exist at a particular pH.

Another means to inhibit proteolytic activity of intestinal chymotrypsin is to employ one or more compounds that bind and inhibit chymotrypsin. Such chymotrypsin inhibitors that may be used in the oral formulations described herein include, but are not limited to, chymostatin, Z-L-phe chloromethyl ketone, α2-antiplasmin, aprotinin (also called “bovine pancreatic trypsin inhibitor” or “BPTI”), 6-aminohexanoic acid, α1-antitrypsin, 4-(2-aminoethyl)benzenesulfonyl fluoride hydrochloride, bromoenol lactone, diisopropyl fluorophosphate, ecotoin, N-acetyl-eglin C, gabexate mesylate, leupeptin trifluoroacetate salt, N-p-tosyl-L-phenylalanine chloromethyl ketone, soybean trypsin-chymotrypsin inhibitor, and combinations thereof.

Agents that inhibit chymotrypsin are also found in a certain plants, including various edible cereals and soybean. Accordingly, extracts, products, or sub-fractions of plants, e.g., rice, soybean, oats, and wheat, may also be present in or administered in conjunction with a formulation described herein to specifically prevent or inhibit proteolytic activity of intestinal chymotrypsin.

The effectiveness of one or more chymotrypsin inhibitors or one or more buffering agents to inhibit chymotrypsin may be initially tested in any standard in vitro enzyme assays for chymotrypsin-mediated proteolytic activity (see, e.g., Examples 7 and 8, below).

Agents for Preventing Degradation of BMP by Intestinal Trypsin

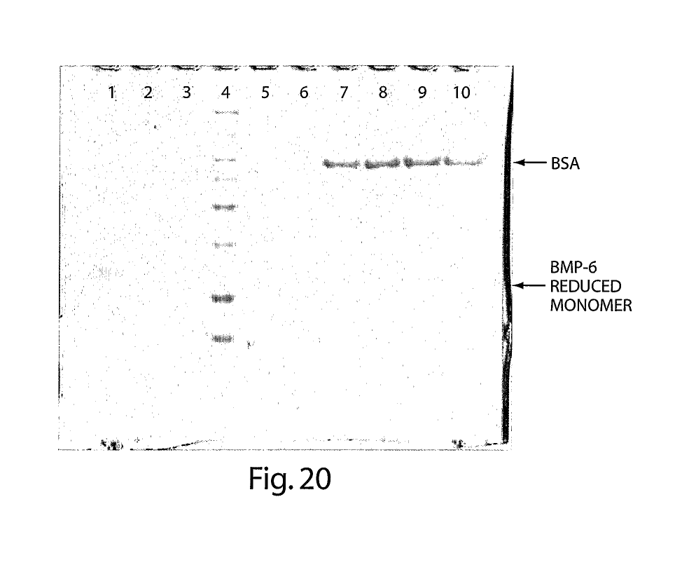

Trypsin specifically hydrolyzes peptides, amides, and esters at lysine (Lys) and arginine (Arg) carboxyl bonds. Trypsin is also an intestinal enzyme with optimal pH of 7.6. Trypsin present in the duodenum appears to be capable of causing a slight truncation of BMP-6 monomeric polypeptides (see, e.g., Example 7 and FIG. 20, below) without significantly affecting the desired osteoinductive pharmacological activity. Nevertheless, it may be desirable to include an agent that inhibits or prevents proteolytic activity of trypsin in an oral formulation comprising a BMP. For example, as even a limited degradation of a BMP may run the risk of greater susceptibility to denaturation or further degradation, use of one or more agents to inhibit or prevent proteolytic activity of intestinal trypsin may be preferred when a BMP is likely to be relatively slowly released into or absorbed from the intestinal tract, e.g., in time released or passive pump preparations. As trypsin is present in the duodenum, agents analogous to those described above for inhibiting chymotrypsin may also be employed in such formulations, i.e., one or more pH lowering agents (buffer) and/or trypsin inhibitors.

A variety of compounds are known that inhibit trypsin proteolytic activity. Such trypsin inhibitors that may be used in formulations and methods of the invention include, but are not limited to, aprotinin (also called “bovine pancreatic trypsin inhibitor” or “BPTI”), α2-antiplasmin, antithrombin III, α1-antitrypsin, antipain, 4-(2-aminoethyl)benzenesulfonyl fluoride hydrochloride, p-aminobenzamidine dihidrochloride, bdellin, benzamidine hydrochloride, diisopropyl fluorophosphate, 3,4-dichloroisocoumarin, ecotin, gabexate mesylate, leupeptin, α2-macroglobulin, phenylmethylsulfonyl fluoride, N-α-p-tosyl-L-phenylalanine chloromethyl ketone, trypsin-chymotrypsin inhibitor, and combinations thereof.

Formulating BMPs for Oral (Enteral) Administration

Various compositions (formulations) may be produced that permit the effective oral (enteral) administration of a BMP, i.e., administration by the mouth or anywhere along the alimentary canal. Generally, a composition that is swallowed must contain one or more agents that protect the BMP from degradation by gastric pepsin and intestinal chymotrypsin. Without intending to be limited to any single composition, an example of an oral formulation useful in the invention may employ an enteric coating to encase, encapsulate, or otherwise sequester a BMP, along with an agent that inhibits the proteolytic activity of duodenal chymotrypsin. Enteric coated formulations may be further encapsulated in any of a variety of pharmaceutically acceptable, dissolvable shells. By way of example, such shells may contain one or more inactive ingredients, such as, gelatin, dyes, titanium dioxide, alkyl alcohols, sodium hydroxide, propylene glycol, shellac, and polyvinyl pyrrolidone. The enteric coated formulation remains intact while passing through the stomach and prevents gastric pepsin and acids from degrading or denaturing the BMP. It may also be desirable to include a specific pepsin inhibitor and/or an antacid (to raise gastric pH), as described above, to provide additional protection from degradation by gastric pepsin. Such additional protection from gastric pepsin may be especially preferred if passage through the stomach may be slower than usual (e.g., due to a filled stomach, effects of other medications, etc.). Upon passage into the duodenum, the enteric coating of the formulation dissolves at the higher pH of the intestinal tract and releases the BMP along with one or more agents that prevent or inhibit proteolytic activity of chymotrypsin. As noted above, the agent that inhibits chymotrypsin proteolytic activity may be a pH lowering agent (e.g., a buffer), a specific inhibitor of chymotrypsin, a plant extract or sub-fraction that inhibits chymotrypsin, or a combination thereof. A pH lowering agent useful in oral formulations described herein may be a buffer, such as one selected from the group consisting of acetate, succinate, lactate, citrate, isocitrate, ascorbate, oxaloacetate, oxalate, malate, fumarate, 2-ketoglutarate, glutarate, pyruvate, glycerate, and combinations thereof. It may be particularly desirable to include both a pH lowering agent and one or more specific inhibitors of chymotrypsin if absorption of the BMP in the intestine is expected to be longer than usual (e.g., as may be the case with a delayed or extended release formulation, a filled intestinal tract, effects of other medications, etc.). Furthermore, although a pH lowering agent is expected to inhibit other intestinal proteases, such as trypsin, it may be desirable to include a trypsin-specific inhibitor, as well, especially if absorption of the BMP from the intestine into the bloodstream is expected to be unusually prolonged or delayed.

It is also understood that if a formulation is to be enterally administered to an individual in a manner that bypasses the stomach, then an agent to prevent or inhibit gastric pepsin proteolytic activity is not a required (i.e., is an optional) component of the formulation. Examples of such formulations include, but are not limited to, a suppository that releases an osteoinductive BMP (or a functionally equivalent osteoinductive protein) into the intestines (e.g., when inserted into the rectum) and a formulation that can be injected directly into the duodenum or colon (e.g., by a single injection or continuously by a pump). In such cases, the concern is for protecting the osteoinductive BMP (or functionally equivalent osteoinductive protein) from degradation by intestinal proteases, especially chymotrypsin, and also for enhancing the absorption of an effective amount of the osteoinductive BMP into the bloodstream. Accordingly, in addition to an osteoinductive BMP (or functionally equivalent osteoinductive protein), such formulations preferably also comprise one or more agents that prevent or inhibit the proteolytic activity of intestinal chymotrypsin and may also comprise one or more agents to prevent or inhibit proteolytic activities of other intestinal proteases, such as trypsin.

Formulations according to the invention may also comprise one or more agents to enhance the absorption of an osteoinductive BMP (or functionally equivalent osteoinductive protein) through the intestinal wall into the bloodstream. An absorption enhancer may be any of a variety of surface active agents or combinations of surface active agents. For example, absorption enhancers useful in formulations of the invention that are administered directly into the intestinal tract include, but are not limited to, anionic agents that are cholesterol derivatives, cationic surface active agents, non-ionic surface active agents, and combinations thereof. Anionic agents that are cholesterol derivatives include bile acids, e.g., cholic acid, deoxycholic acid, taurocholic acid, taurodeoxycholic acid, fusidic acid, glycholic acid, dehydrocholic acid, lithocholic acid, ursocholic acid, ursodeoxycholic acid, and the like. Cationic surface active agents include acylcarnitines, acylcholines, lauroylcholine, cetyl pyridinium chlorides, cationic phospholipids, and the like. Non-ionic surface active agents include polyoxyethylene ethers (e.g., BRIJ non-ionic detergents), p-t-octyl phenol poloxyethylenes (e.g., TRITON X-100 non-ionic detergents), nonylphenoxypoloxyethylenes, polyoxyethylene sorbitan esters, and the like.

Depending on the site of administration along the intestinal tract, a formulation of the invention may also comprise a pH lowering agent and/or one or more specific inhibitors of intestinal proteases as discussed above, especially if the formulation will pass through the duodenum where significant levels of chymotrypsin are found. Suppositories that are administered to the lower portion of the colon (i.e., via the rectum) also comprise a pH lowering agent and/or one or more specific inhibitors effective against duodenal chymotrypsin and, optionally, other proteases (e.g., trypsin), as such enzymes may pass into or otherwise be found in the colon of individuals.

Assessment of Bones of Patients of Metabolic Bone Disorders

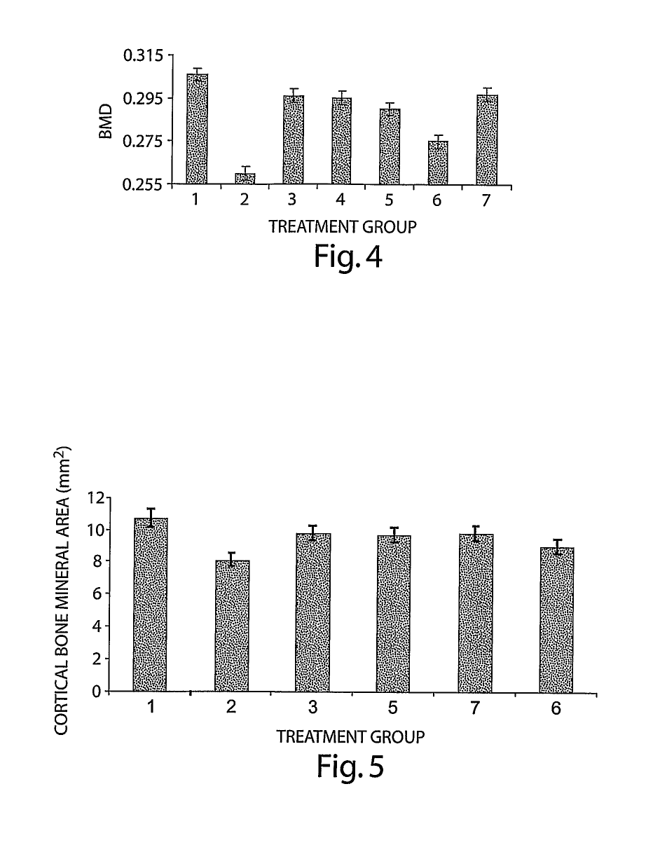

As noted above, evidence of a metabolic bone disorder, such as osteoporosis, is often only detected once the disorder has advanced to the point that a fracture, e.g., in the wrist, spine, or hip, is presented clinically. The condition of the bones, bone mineral density (BMD), and/or bone mineral content (BMC) of an individual may be assessed by any of a number standard methods known in the art including, without limitation, traditional X-rays, radiographic absorptiometry, magnetic resonance imaging (MRI), and, more recently developed, dual energy X-ray absorptiometry (DEXA) analysis. DEXA analysis provides a particularly accurate, non-invasive analysis of BMD of the bones of an individual and, thus, is a particularly preferred method for diagnosing even relatively early stages of progressive metabolic bone disease, such as osteopenia and osteoporosis, and for monitoring enhancement or restoration in BMD using methods and compositions described herein (see, e.g., Example 1 and FIG. 4, below).

Additional Considerations for Therapeutic Compositions and Methods

Methods of the invention for treating a metabolic bone disorder characterized by loss of bone growth may comprise administering to the individual an effective amount of an osteoinductive BMP in combination with one or more agents that prevent or inhibit proteolytic activity of gastric pepsin and/or one or more agents that prevent or inhibit proteolytic activity of duodenal chymotrypsin. Preferably, an oral formulation comprising a BMP and agents for preventing or inhibiting proteolytic activity of particular gut enzymes is swallowed by an individual and passes through the stomach and into the intestinal tract where an effective amount of the BMP is released for absorption into the bloodstream. It is also possible, however, that in some situations such agents and/or BMP may be administered directly at a point along the alimentary canal, e.g., a suppository or using active or passive pumps that can inject a composition or agent(s) locally as in the stomach or intestinal tract. Oral administration of a formulation of the invention may also be administered with the assistance of a mechanical device such as a nasojejunal or gastrostomy tube that is inserted into an individual.

As noted above, agents to prevent or inhibit proteolytic activity of one or more gut enzymes may be present in the same composition as a BMP, however, it is also possible that such agents may be delivered sequentially or concurrently as separate compositions provided the desired protection of the BMP from proteolytic degradation of gut enzymes is sufficient to permit an effective amount of the BMP to reach the intestines and to be absorbed into the bloodstream.

It is also understood that oral formulations of the invention may further comprise one or more additional therapeutic compounds that provide one or more additional pharmacological benefits or activities in addition to the osteoinductive activity of the BMP present in the formulation. Such additional therapeutic compounds should not significantly diminish the desired osteoinductive activity of the orally administered BMP.

The optimal amounts of agents present in formulations described herein to inhibit or prevent proteolytic activities of various gut enzymes may be determined by routine analytical procedures employed by persons skilled in the art of pharmaceutical formulations.

Dosing for a particular individual (patient) who has, is suspected of having, or is at risk of having a metabolic bone disorder, such as osteoporosis, will be determined by the attending skilled healthcare provider taking into account a variety of clinical parameters that characterize that patient, e.g., ability to swallow, age, gender, weight, possible genetic factors, evidence of one or more other diseases, and the like. By way of non-limiting example, a BMP, such as BMP-6, may be orally administered to an individual at a dose in the range of from 0.5 mg/day to 5 mg/day. Thus, doses of 0.5 mg/day to 5 mg/day may be used in compositions and methods described herein. A particularly useful dose to initiate treatment and which may also be maintained during a course of treatment is 0.5 mg/day of BMP. Furthermore, a BMP may be administered periodically or cyclically to an individual, e.g., administration to an individual for a period of time, discontinued for a period for time, and then re-initiated. The limitation on a course of dosing or repetition of dosing typically will be based on whether the attending healthcare provider believes such dosing or repetition may or may not provide further benefit to a particular individual and/or whether there is any evidence of acute or chronic side effects that would limit the use of a particular dose or duration of administering BMP orally to the individual.

It is also understood that persons skilled in the art are aware that doses of pharmacologically active compounds, such as a BMP, may be expressed not only in terms of mass, e.g., micrograms (μg) or milligrams (mg), of drug administered per day, but other units as well as, including, but not limited to, an amount of BMP per kilogram of body weight or mass of an individual (e.g., μg/kg, mg/kg), amount per surface area (e.g., μg/m², mg/m²), mg per unit volume (e.g., per mL) of formulation, and the like. As used herein, discussion of dosages for humans in terms of mg/day refer to mg per individual per day and are based on the commonly used standard of a 70 kg male human patient. Similarly, discussions of dosing for humans in terms of mg of compound per kg of body weight (mass) assume a 70 kg male human being. It is understood, therefore, that doses may have to be modified for a particular individual or population of individuals. For example, this is particularly relevant in the case of osteoporosis. Approximately 80% of individuals diagnosed with osteoporosis are post-menopausal women; many of whom weigh less than 70 kg and may be relatively frail in weight and bone structure compared to a healthy individual (male or female). Hence, it is understood that when treating an individual that is more or less than 70 kg, a dose may be appropriately modified in accordance with standard pharmacological adjustments. Accordingly, various examples of doses described herein are readily converted by persons skilled in the art to various other dosing units (and vice versa) required for treating specific individuals or populations of individuals with a particular oral formulation comprising a BMP as described herein.

As mentioned throughout this description of the invention, the oral formulations of BMP that are to be swallowed or otherwise administered to the stomach of an individual must also comprise one or more agents that prevent proteolytic degradation of BMP by gastric pepsin and by intestinal chymotrypsin. Such formulations may further comprise an agent that inhibits intestinal trypsin as well. It is also understood that if a formulation is administered to an individual directly via the intestines, e.g., suppositories or injection into the intestines, an agent to inhibit gastric pepsin activity is not required, i.e., is an optional component.

In addition to the above-mentioned useful agents that prevent degradation of BMPs in the stomach and intestines, more generally, compositions of the invention may be formulated for administration by an enteral route to an individual according to standard pharmaceutical protocols and texts (e.g., Remington's Pharmaceutical Sciences, 18th ed., Alfonso R. Gennaro, ed. (Mack Publishing Co., Easton, Pa. 1990)). The compositions of the invention comprising an osteoinductive BMP (or functionally equivalent osteoinductive protein) for oral (enteral) administration may be prepared in any of a variety of dosage forms including, but not limited to, tablets, mini-tablets, capsules, granules, powders, effervescent solids, chewable solid tablets, softgels, caplets, aqueous solutions, suspensions, emulsions, microemulsions, syrups, or elixirs. In the case of tablets for oral use, carriers, which are commonly used, include lactose and corn starch. Lubricating agents, such as magnesium stearate, may also be added. Some dosage forms, including, but not limited to, capsules, tablets, pills, and caplets, may also be particularly well suited for formulations that provide delayed, extended, or sustained release of BMP to the intestinal tract of an individual. If desired, certain sweetening and/or flavoring and/or coloring agents may also be added.

Thus, a composition comprising a BMP and one or more agents to inhibit or prevent proteolytic activity of gastric pepsin and/or duodenal chymotrypsin may also comprise any of a number of various pharmaceutically acceptable buffers or carriers, excipients, or adjuvants known in the art that may provide one or more beneficial properties, including but not limited to, more efficient or less painful administration to an individual (e.g., to enhance combination of ingredients, ease of swallowing, ease of injection, ease of insertion), more efficient or time-released delivery of a BMP in the intestinal tract of an individual, and/or stability for longer storage of compositions (i.e., enhanced shelf-life). Accordingly, pharmaceutical compositions of this invention may further comprise any of a number of compounds that may be employed in formulations for enteral delivery including, by not limited to, water, ion exchangers, alumina, aluminum stearate, lecithin, serum proteins, such as human serum albumin, buffering compounds (e.g., acetates, phosphates, glycine), sorbic acid, potassium sorbate, partial glyceride mixtures of saturated vegetable fatty acids, and salts or other electrolytes (e.g., sodium chloride, protamine sulfate, disodium hydrogen phosphate, potassium hydrogen phosphate, zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, fats, and combinations thereof.

Additional embodiments and features of the invention will be apparent from the following non-limiting examples.

### EXAMPLES

When a dose of BMP-6 administered to an animal in the studies below is indicated as a particular amount “per kilogram” (“/kg”), it is understood that such designation means “per kilogram of body weight” of the individual animal.

### Example 1

### Dose Response and Efficacy of Intravenously Administered Bone Morphogenetic Protein-6 (BMP-6) in Aged Ovariectomized Rats

This study shows that intravenous administration of BMP-6 is effective in promoting bone growth in a rat model of osteoporosis.

Materials and Methods

Animals and study protocol. One hundred sixty (160), 4 months old Sprague-Dawley female rats were used in this study. Animals weighed approximately 300 grams. The rats were kept in standard conditions (24° C. and 12 hour/12 hour light/dark cycle) in 20×32×20 cm cages during the study. All animals were allowed free access to water and pelleted commercial diet (Harlan Teklad, Borchen, Germany) containing 1.00% calcium, 0.65% phosphorus, and 2.40 KIU of Vitamin D3 per kilogram. Estrogen was administered as estradiol. Recombinant BMP-6 was prepared from transfected CHO cells following standard procedures.

On Days-14 and -4, animals received calcein green labeling regimen (15 mg/kg, intraperitoneally, i.p.), which resulted in the deposition of double fluorochrome labels on active bone forming surfaces.

Forty (40) animals were sham operated while the rest were ovariectomized (OVX) bilaterally by abdominal approach. Treatment started twelve months after ovariectomy as follows:

| | |
|---|---|
| Group 1. | SHAM |
| Group 2. | OVX control, treated with vehicle (acetate buffer, 3 times |
| | per week, i.v.) |
| Group 3. | OVX treated with BMP-6 (3 times per week, at 10 μg/kg, |
| | intravenously, i.v.) |
| Group 4. | OVX treated with BMP-6 (3 times per week, at 25 μg/kg, |
| | i.v.) |
| Group 5. | OVX treated with BMP-6 (3 times per week, at 50 μg/kg, |
| | i.v.) |
| Group 6. | OVX treated with estradiol-E2 (175 μg/week, |
| | administered in 3 doses per week at 50, 50, and 75 μg/rat, |
| | subcutaneously, s.c.) |
| Group 7. | OVX treated with estradiol + BMP-6 (estradiol: 175 μg/ |
| | week, administered 3 times per week, at 50, 50, and 75 μg/ |
| | rat, s.c.; and BMP-6 administered at 10 μg/kg, i.v.) |

Animals were treated for 12 weeks.

Bone mineral monitoring in vivo. Animals were scanned prior to ovariectomy, at three months after ovariectomy, two times during therapy, after 6 weeks of treatment, and after 12 weeks of treatment using dual energy absorptiometry (DXA, HOLOGIC QDR-4000, Hologic Inc., Waltham, Mass., U.S.) equipped with Small Animal software. Prior to scanning, animals were anesthetized with thiopental barbiturate (Nycomed Pharma GmbH, Ismaning, Germany).

Total body scans were performed. Bone mineral density (BMD) and bone mineral content (BMC) of lumbar vertebrae, hind limbs, total body, and total body with head excluded were determined.

Prior to sacrificing animals for analysis, urines were collected. For urine collection animals were placed in metabolic cages and deprived of food for an overnight period of 18 hours. Sacrifice started 12 weeks after the beginning of therapy by ether anesthesia.

Bone mineral measurements ex vivo. After sacrifice femora, tibiae, and lumbar vertebrae were harvested and scanned BMD and BMC of whole left femora, distal femoral metaphyses (the second 0.5 cm from the distal end of femur) and proximal end of femur (with femoral head, neck, and great trochanter included) were measured.

pQCT analysis of femurs. Rat bones were additionally analyzed using a pQTC scanner (Stratec-Norland, Medizintechnik, Pforzheim, Germany), which enables a precise quantitative analyses based on computerized tomography. pQCT analysis is primarily used for measurements of cortical bone.

MicroCT analysis of femurs. Femurs were scanned with microCT machine (Skyscan, Aartsclaar, Belgium), which reconstructs three-dimensional image of 4 μm thick slices. MicroCT measurements enable analysis of trabecular bone and microarchitecture thereof.

Biomechanical testing. The effect of BMP-6 on the mechanical properties of bone was investigated by indentation test of the distal femoral metaphysis (DFM) and by three-point bending test of femoral shaft. The indentation test was used to determine the mechanical properties of cancellous bone in the marrow cavity of the DFM. The three-point bending test was used to determine the mechanical properties of the midshaft femur.

Femoral bone histomorphometra. The right femora, tibia, and lumbar vertebrae were taken for histomorphometry. Femurs were fixed in 70% alcohol and embedded in methacrylate. Goldner staining was preformed on sections of distal femurs (4 mm proximal to condyles). Static and dynamic histomorphometry was performed on the sections.

Results

In vivo Bone Mineral Density (BMD)

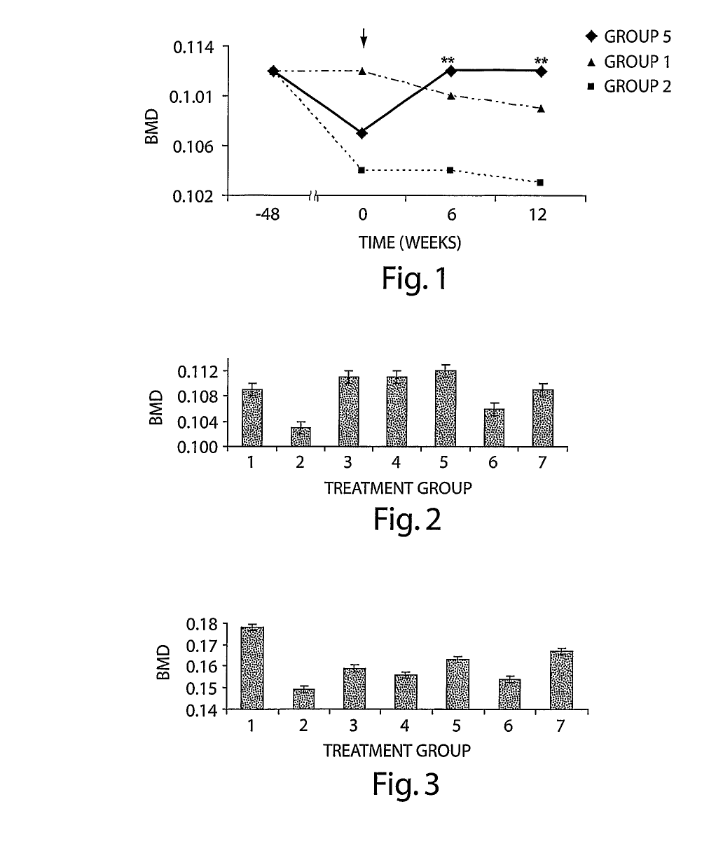

Twelve months following ovariectomy, hind limb bones of the rats lost about 6% of BMD as compared to sham treated animals (see, in FIG. 1). All doses (i.e., 10, 25, 50 μg/kg, i.v.) of BMP-6 were potent in restoring lost BMD, with BMP-6 at 50 μg/kg, i.v., yielding the most significant restoration of BMD (see, Group 5 in FIG. 1). Within 6 weeks of treatment, BMP-6 administered rats regained the lost BMD and, at 12 weeks following treatment, had significantly better BMD as compared to both Group 2 untreated, ovariectomized animals (P<0.0001) and Group 1, sham animals (P<0.02) (see, FIG. 1). Sham treated rats lost about 2% of their BMD within the same time period of 12 weeks following therapy with BMP-6. Rats treated with estradiol alone (Group 6) had increased BMD values, but not significantly compared to ovariectomized control animals (compare Group 6 with Group 2 in FIG. 2). When BMP-6 was added to estrogen-treated rats (Group 7), BMD values were already improved after 6 weeks, reaching significantly higher BMD values at 12 weeks following therapy, although significantly less than any group treated with BMP-6 alone (see, FIG. 2).

At the level of spine, all doses of BMP-6 were efficacious but did not reach the sham BMD values as at hind limbs. BMP-6 administered at a dose of 50 μg/kg increased BMD values at both 6 and 12 weeks of treatment compared to Group 2 ovariectomized control animals (P<0.0001). At 12 weeks, the data indicated that about 50% of the lost BMD was regained (see, FIG. 3). A longer treatment period would be needed to restore the lost bone at the vertebral bone envelope. Rats treated with estrogen alone (Group 6) showed increased BMD values at 6 weeks in a range similar to rats treated with estrogen and BMP-6 (Group 7). At 12 weeks, BMD values of estrogen-treated rats (Group 6) were not different from ovariectomized animals treated with vehicle (acetate) buffer only (Group 2, see, FIG. 3). These results indicated that estrogen alone could not maintain the gains in BMD if not combined with BMP-6.

In summary the in vivo hind limb BMD at three months of therapy with intravenously administered BMP-6 and estrogen showed that all three doses were effective and capable of regaining the lost BMD; the highest dose resulting in the highest gain in BMD (Group 5, FIG. 2). Estrogen alone (Group 6) had no effect unless combined with BMP-6 (as in Group 7). Moreover, in spine, BMP-6 at 10 μg/kg was synergistic to estrogen alone values (compare Group 7 with Group 6, FIG. 3). Following termination of the study ex vivo uterine weights were recorded to exclude uterus as a target for BMP-6. As expected, only rats treated with estrogen alone or with estrogen and BMP-6 had increased uterine weights.

BMD ex vivo. BMD values of tibias, femurs, and spines of rats were determined ex vivo. Highly significant BMD gain was recorded in the femurs and tibias of rats treated with BMP-6, independently of a dose used (see, e.g., femur data in FIG. 4), with P values in the range of 10⁻⁶ to 10⁻⁹. Administering estrogen alone (Group 6) also increased the BMD values, but at a 2 to 3 times lower level as compared to BMP-6 treated animals.

pQCT analysis. A pQCT analysis of femurs showed that total BMD was higher in all BMP-6 treated rats as compared to ovariectomized control animals. Moreover, rats receiving estrogen had about 8% higher BMD values than ovariectomized control (Group 1, OVX) animals. Rats treated with 10 μg of BMP-6 (Group 3) showed 13.8% higher BMD than control animals. Total femoral bone mineral content was about 18% higher in BMP-6 treated rats, and 11.5% in estrogen treated rats, which was statistically significant using a rigorous ANOVA/Dunnett-test analysis.

Subdividing further individual bone components, the data revealed that BMP-6 influenced primarily the bone area and mineral content of the cortical region. Cortical bone mineral content was 24% higher in all BMP-6 treated rats as compared to ovariectomized control animals (P<0.0001), and the cortical bone mineral area (mm²) was about 21% above the control values (see, FIG. 5).

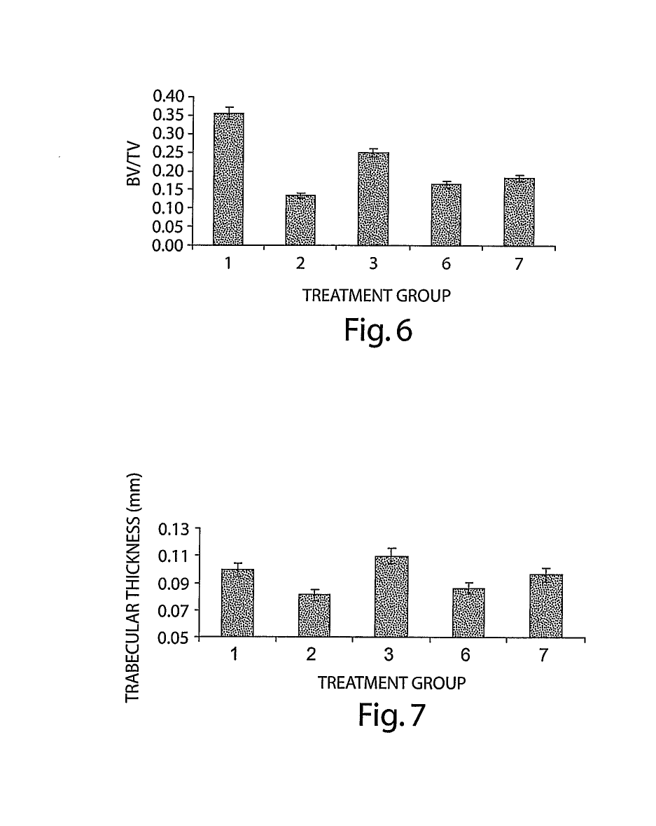

MicroCT analysis. MicroCT analysis of femurs showed that the ratio of bone volume/trabecular volume (BV/TV) was significantly higher in BMP-6 treated rats (Groups 3-5) as compared to ovariectomized control animals (Group 2), estrogen treated rats (Group 6), and estrogen +BMP-6 treated rats (Group 7). Moreover, rats treated with 10 μg of BMP-6 (Group 3) had 82.3% increase in BV/TV values compared to ovariectomized control animals (Group 2) and 46.9% increase compared to rats receiving estrogen (Group 6) (see, FIG. 6). Trabecular number was 34.8% higher in BMP-6 treated rats than in ovariectomized control rats, and 14.3% higher than in estrogen treated rats, which was statistically significant using a rigorous ANOVA/Dunnett-test analysis. Trabecular thickness showed statistically significant increase in values of BMP-6 treated rats (e.g., Group 3, 10 μg of BMP-6) compared to ovariectomized control animals (Group 2), estrogen-treated (Group 6), and estrogen+BMP-6 treated (Group 7) rats. BMP-6 treated animals of Group 3 even had 10.5% higher trabecular thickness than sham animals of Group 1 (see, FIG. 7).

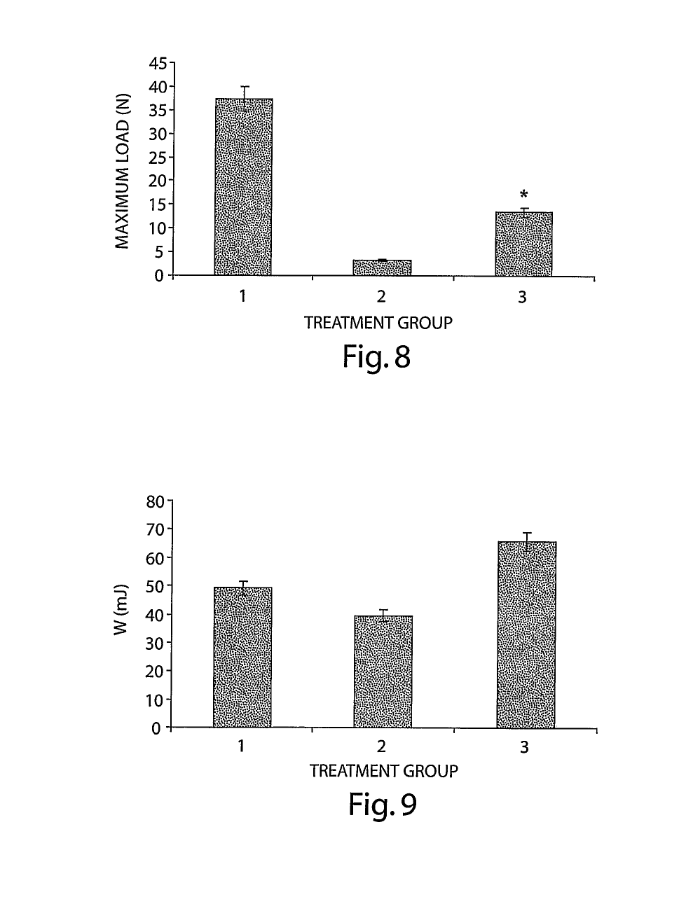

Biomechanical testing. The indentation test was used to determine the mechanical properties of cancellous bone in the marrow cavity of the distal femoral metaphysis (DFM). Direct parameters: maximal load, stiffness, and energy absorbed were increased 3-fold in BMP-6 treated rats (e.g., Group 3 animals) compared to ovariectomized control animals (Group 2), which was statistically significant (see, FIG. 8). Ultimate strength (derived parameter) showed the same trend.

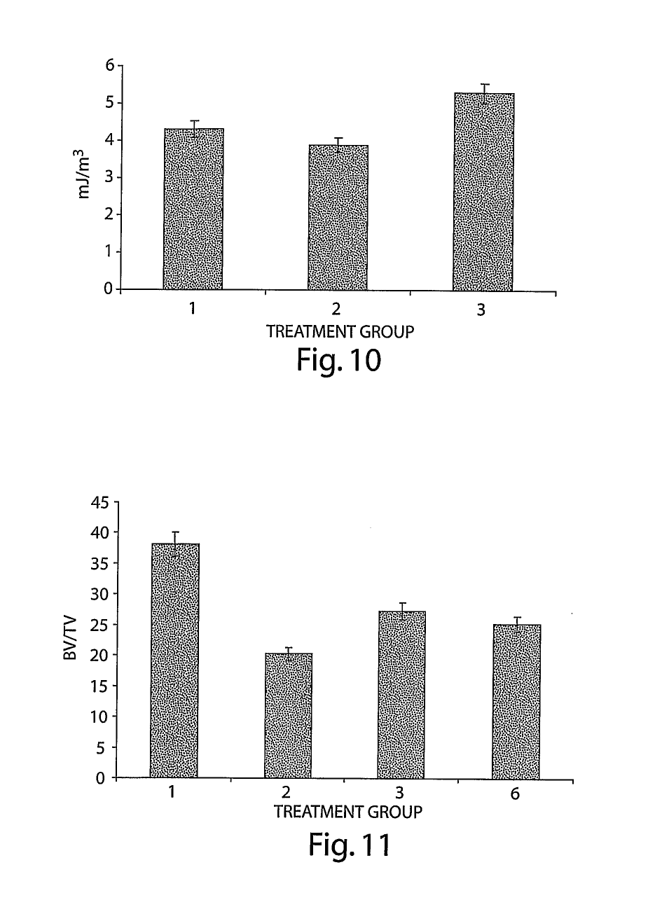

The three-point bending test was used to determine the mechanical properties of the midshaft femur. Maximal load and stiffness were significantly higher in BMP-6 treated animals (e.g., Group 3) compared to ovariectomized control rats (Group 2). Bones from BMP-6 treated-animals absorbed 33.4% more energy (i.e., Work: “W”, expressed in millijoules) than sham animals (Group 1, P<0.05) (see, FIG. 9). Toughness (a derived parameter measured as millijoules (mJ)/m³) of BMP-6 treated animals was increased 22.3% compared to sham animals (Group 1) and showed statistical significance (see, FIG. 10).

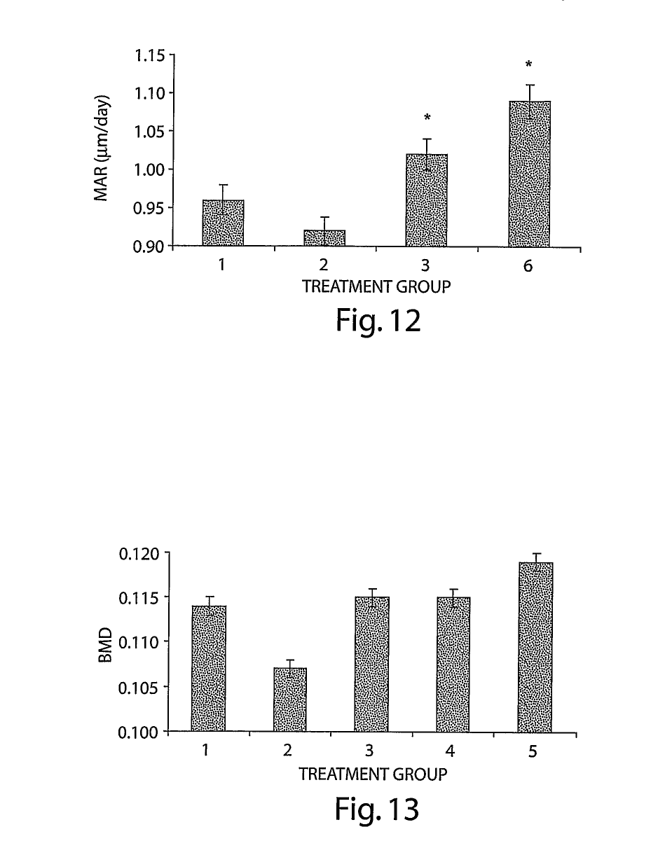

Histomorphometry. Histomorphometry (a computerized procedure of measuring via microscopy bone parameters in tissue sections) confirmed pQCT and microCT analyses. Bone volume/trabecular bone volume (BV/TV) of distal femurs was significantly higher in BMP-6 treated animals (e.g., Group 3) as compared to ovariectomized rats treated with vehicle buffer (Group 2; see, FIG. 11). Dynamic histomorphometry (a procedure that measures incorporation of tetracycline into bone using fluorescent microscopy) showed increased mineral apposition rate (“MAR”, μm/day) in BMP-6 treated rats (e.g., Group 3) or rats treated with estrogen+BMP-6 (Group 6) that was statistically significant (P<0.001) as compared to Group 2 ovariectomized control rats treated with vehicle buffer (see, FIG. 12).

Conclusion

BMP-6 administered intravenously significantly increased the BMD, both in vivo and ex vivo in aged, ovariectomized rats over a period of 12 weeks. pQCT analysis showed great influence of the BMP-6 on the cortical bone. In addition, microCT analysis showed increased trabecular thickness in BMP-6 treated rats, which reached sham values. This is of particular interest since there is no known agent to have been previously reported to fully restore lost bone in aged, ovariectomized rats. The only agent previously reported to have an anabolic bone effect is parathyroid hormone, which acts on both trabecular and cortical bone, however, its action on the cortex also produces bone resorption tunnels. Such resorptive tunnels weaken the mechanical characteristics of the bone and in the long term could have a deleterious effect on its mechanical properties. Biomechanical testing showed statistically significant improvement of mechanical properties of bones from BMP-6 treated animals compared to ovariectomized control animals, even being tougher than bones from sham animals.

### Example 2

### Effect of Lower Doses of BMP-6 on Bones in Aged, Ovariectomized Rats

Seven-month old Sprague-Dawley rats were ovariectomized (OVX), as above, and were left for approximately 20 months to lose bone mineral density (BMD). Thus, therapy was initiated 72 weeks following ovariectomy, at the age of 2 years and 1 month and continued for 3 months, until the sacrifice for analysis. Animals were divided into following groups:

| | |
|---|---|
| Group 1. | SHAM (n = 8) |
| Group 2 | OVX control (n = 8) |
| Group 3 | OVX treated with BMP-6, 10 μg/kg, 3 × week (n = 8) |
| Group 4 | OVX treated with BMP-6, 10 μg/kg, 1 × week (n = 12) |
| Group 5 | OVX treated with BMP-6, 1 μg/kg, 3 × week (n = 12) |

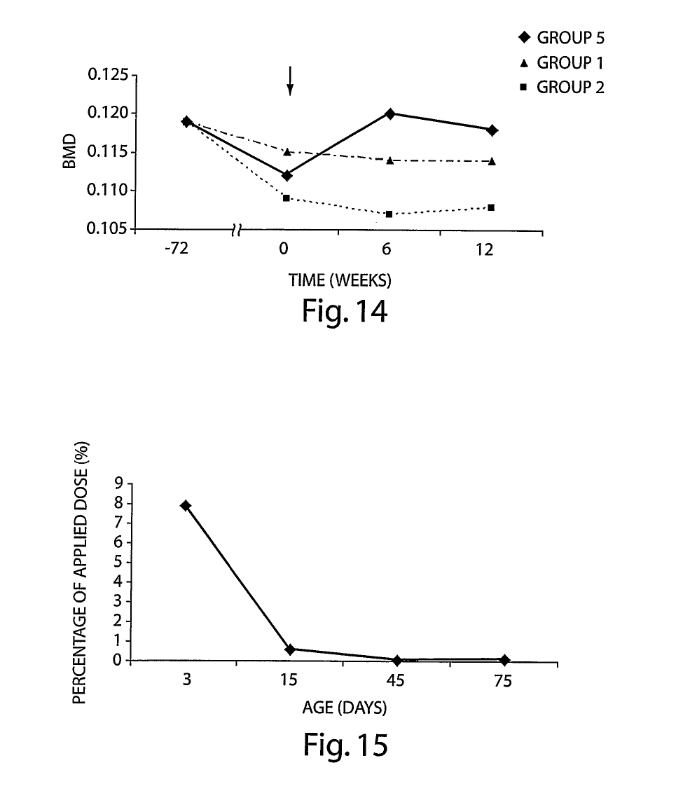

BMD in vivo. In vivo BMD was monitored every 6 weeks. At 6 weeks following the initiation of therapy, all BMP-6 treated animals showed statistically significant higher BMD values of hind limbs as compared to OVX control animals, even having higher BMD than sham animals. There were no statistically significant differences between BMP-6 treated groups. BMP-6 at doses of 1 μg/kg, three times per week (Group 5), increased BMD of hind limbs for 11.2% in comparison to OVX animals (Group 2), while BMP-6 at doses of 10 μg/kg, once (Group 4) and three times (Group 3) per week, increased BMD for 7.6% (see, FIG. 13). At 12 weeks following the beginning of therapy, BMP-6 treated animals (Group 5) retained high BMD values even in comparison with sham animals (Group 1), but lost some bone in comparison to earlier measurement on the 6th week. This phenomenon could be explained by the aging of the animals, since only a few animals can survive to 2 years and 7 months, the time when the experiment was terminated (see, FIG. 14).

Conclusion

Low, intravenously administered, doses (e.g., 1 μg/kg, 3 times per week, i.v.) of BMP-6 are even more effective in restoring lost bone than higher doses over the time period of 12 weeks. In addition, BMP-6 at a dose of 10 μg/kg, once weekly, is as effective on BMD as BMP-6 administered three times per week.

### Example 3

### Duodenal Absorption and Biodistribution of BMP-6 Labeled with 99m Technetium

This study shows that the efficacy of orally administered BMP-6 for inducing bone formation in an individual can depend on the age of the individual. In particular, bone morphogenetic proteins degrade under the influence of gastric enzymes that are known to be present in adults, but typically not in infants. Accordingly, this study was undertaken to compare the quantity of orally (via mouth) and duodenally administered BMP absorbed in infant and adult individuals. Specifically, the absorption of labeled BMP-6 was compared rats that were 3 days old, 15 days old, 45 days old, and 75 days old.

BMP-6 labeling. Mature BMP-6 was chelated with mercaptoacetylthreeglycin (MAG3). BMP-6-MAG3 complex was labeled with radioactive 99m Technetium-pertechnetate (99mTc). Chromatography revealed that more than 97% of 99mTc was ligated to the complex.

Animals and therapeutic protocol. Animals were divided into the following treatment Groups:

| | |
|---|---|
| Group 1. | 3 days old. 100 μg/kg BMP-6 labeled with |
| | 99mTc, applied with pipette directly into |
| | the mouth. |
| Group 2. | 15 days old. 100 μg/kg BMP-6 labeled |
| | with 99mTc, applied with pipette directly |
| | into the mouth. |
| Group 3. | 45 days old. 100 μg/kg BMP-6 labeled |
| | with 99mTc applied with syringe directly |
| | to duodenum. |
| Group 4. | 75 days old. 100 μg/kg BMP-6 labeled |
| | with 99mTc applied with syringe directly |
| | to duodenum. |

Intraduodenal application. Animals were anesthetized with thyopenthal and were subjected to abdominal surgery. After revealing of abdominal organs and isolation of the duodenum, BMP-6 labeled with 99mTc was injected with syringe and needle directly into duodenum.

Measurement of radioactivity with gamma counter. Animals were sacrificed 60 minutes after surgery. Blood and all organs were taken for measurement. All samples were measured for the amount of radioactivity with gamma counter and were expressed as counts per minute (cpm). The results were expressed as a percentage of applied dose, comparing the measured radioactivity with radioactivity of a standard that had the same radioactivity as the total applied dose. All values were corrected in dependence of the half-life factor.

Results

Three-day old animals absorbed 9% of the applied dose, while 15-day old animals absorbed only 0.5% of the applied dose. Older animals, i.e., 45-day and 75-day old, absorbed only 0.1% of applied dose (FIG. 15). These results suggest that infants that do not have developed gastric enzymes can absorb greater amounts of BMP-6 when applied orally than adults.

Effect of pH Lowering Agent and Enhancers on Duodenal Absorption of BMP-6

In light of the above findings, a further study was performed with adult rats and agents known to modify the gastrointestinal environment. Rats that were 60 days old and weighing approximately 200 g were subjected to intraduodenal (i.d.) application of the following substances:
Animal 1. “pH3”
(BMP-6-MAG-3)=166 μL
(acetate buffer 0.1M, pH 2.5)=498 μL
Total volume=664 μL
Final pH 3, as measured by Sigma brand pH test strips, pH range=0.0-6.0 (St. Louis, Mo.)
Animal 2. “pH 3+enhancer”
(BMP-6-MAG-3)=166 μL
(acetate buffer 0.1M, pH 2.5)=498 μL
Total volume=664 μL
Final pH 3, as measured by pH test strips
Addition of enhancers:
   1 mg taurodeoxycholic acid sodium
   1 mg DL-lauroylcarnitine chloride
Animal 3. “enhancer”
(BMP-6-MAG-3)=166 μL
(0.9% NaCl, pH 7)=498 μL
Total volume=664 μL
Addition of enhancers:
   1 mg taurodeoxycholic acid sodium
   1 mg DL-lauroylcarnitine chloride

At 60 minutes following surgery, animals were sacrificed, and samples were taken for measurement of radioactivity.

Results

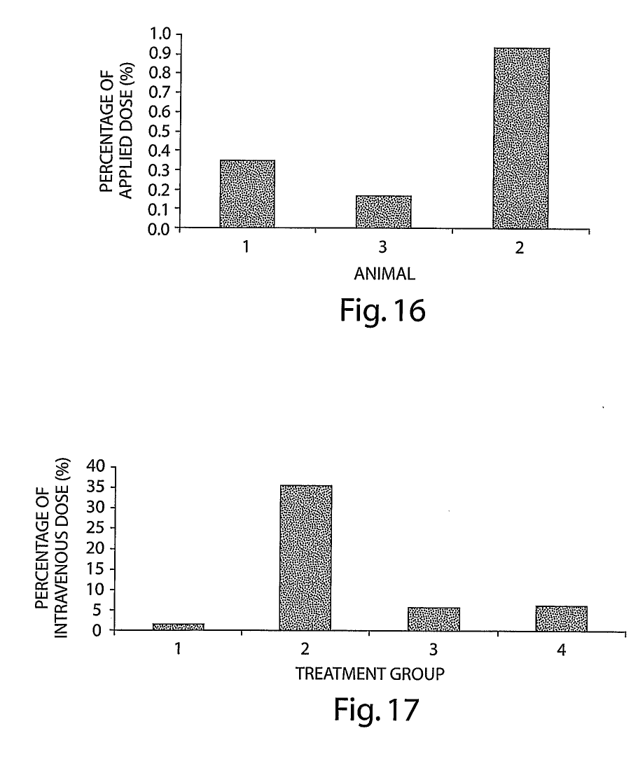

Addition of acetate buffer (pH 3) to BMP-6 (animal 1) resulted in an absorption of BMP-6 of 0.38% of the applied dose, i.e., about 4-fold higher than in the absence of acetate buffer. Application of both acetate buffer (pH 3) and absorption enhancers, i.e., taurodeoxycholic acid sodium and DL-lauroylcarnitine chloride (animal 2), increased duodenal absorption of BMP-6 to 0.94%, while addition of absorption enhancers alone (animal 3) enabled absorption of only 0.16% of applied dose (FIG. 16). These results suggest that BMP-6 can be absorbed through the gastrointestinal system, under proper conditions. Since relatively low doses of BMP-6 act as effectively on osteoinduction as higher doses (see, above), the data suggest that a dosage formulation that enables even a small improvement in the percentage of BMP-6 to pass through the duodenum of an adult could be sufficient for the systemic action on bone formation.

### Example 4

### Effect of Duodenal Application of BMP-6 on Bone Formation in Subcutaneous Bone Pellet (Matrix)

This study shows that duodenally applied and absorbed BMP-6 is active for induction of bone formation. Demineralized and extracted bone matrix implanted subcutaneously is used as a surrogate marker of bone formation. The value and limit of this assay is that evidence of local bone formation in an implanted bone matrix after administration of a growth factor, such as a BMP, at a particular site (e.g., the duodenum) is considered as one indication that the growth factor is capable of acting systemically from that site in the presence of the components of the particular formulation.

Bone pellet. Donors for bone pellet preparation were 20-week old Sprague-Dawley rats. After sacrifice, diaphyses of femurs and tibias were taken for making the pellet. Bones were prepared with addition of chloric acid and urea. In a subcutaneously implanted bone pellet, there is no spontaneous formation of new bone.

Animals and treatment protocol. Sprague-Dawley rats, weighing approximately 200 g, were subjected to surgery. Bone pellets were implanted subcutaneously into the axillar region. Animals were divided into the following treatment Groups, with 4 pellets implanted per group:

| | |
|---|---|
| Group 1. | Control animals |
| Group 2. | Animals receiving BMP-6 at doses of 5 μg/kg |
| | intraduodenally (i.d., as described above) |
| Group 3. | Animals receiving BMP-6 at doses of 50 μg/kg, i.d. |
| Group 4. | Animals receiving BMP-6 at doses of 500 μg/kg, i.d. |
| Group 5. | Animals receiving BMP-6 at doses of 1000 μg/kg, i.d. |
| Group 6. | Animals receiving BMP-6 at doses of 10 μg/kg, i.v. |

Animals of treatment Groups 2-5 were injected twice with the appropriate dose of BMP-6, as described above, directly into the duodenum: 6 hours and 25 hours following implantation of bone pellet. Intravenous (i.v.) application was performed at 6, 12, 24, and 42 hours after surgery. Animals were sacrificed 15 days after surgery, and bone pellets were taken for histology.

Histology. Bone pellets were fixed in 70% alcohol, decalcified, and embedded in paraffin. Sections were stained with toluidine blue. Pellets were considered positive in the presence of new bone formation.

Results

Results are shown in Table 1, below. Animals receiving BMP-6 intraduodenally (i.d.) showed new bone formation (osteoinduction) in subcutaneous bone pellets, suggesting that BMP-6, under proper conditions, can pass the gastrointestinal system in sufficient amount for systemic action on bone formation. Control animals did not show signs of osteoinduction in bone pellets.

TABLE 1.

| Group | Bone Formation |
|---|---|
| (BMP-6 dose) | (Positive/Implanted Pellet) |
| Control | 0/4 |
| (0 μg/kg) | |
| Group 2 | 4/4 |
| (5 μg/kg, i.d.) | |
| Group 3 | 3/4 |
| (50 μg/kg, i.d.) | |
| Group 4 | 4/4 |
| (500 μg/kg, i.d.) | |
| Group 5 | 4/4 |
| (1000 μg/kg, i.d.) | |
| Group 6 | 4/4 |
| (10 μg/kg, i.v.) | |

### Example 5

### Duodenal Absorption of BMP-6 Labeled with 99mTc Compared to Intravenously Applied BMP-6

This study compares the absorption of BMP-6 as a function of duodenal and intravenous administration.

BMP-6 labeling. Mature BMP-6 was chelated with mercaptoacetyl-3-glycin (MAG3). BMP-6-MAG3 complex was labeled with radioactive 99mTechnetium-pertechnetate (99mTc). Chromatography revealed that more than 97% of 99mTc was ligated to the complex.

Animals and therapeutic protocols. Six Sprague-Dawley rats, weighing approximately 200 g entered the experiment. Animals were divided into treatment Groups having the following therapeutic regimens:

Group 1 (n=1)

Vol (BMP-6-MAG-3)=200 μL
Vol (acetate buffer 20 mM, pH 4.0)=400 μL
Total Vol=600 μL
Administration Route: Intraduodenally (i.d.)
   Group 2 (n=2)
Vol (BMP-6-MAG-3)=200 μL
Vol (acetate buffer 0.1 M, pH 3.0)=400 μL

Total Vol=600 μL

| | |
|---|---|
| Addition of enhancers: | 1 mg taurodeoxycholic acid sodium |
| | 1 mg DL-lauroylcarnitine chloride |

Administration Route: i.d.
   Group 3 (n=1)
Vol (BMP-6-MAG-3)=200 μL
Vol (acetate buffer 0.1M, pH 3.0)=400 μL

Total Vol=600 μL

| | |
|---|---|
| Addition of enhancers: | 1 mg taurodeoxycholic acid sodium |
| | 1 mg DL-lauroylcarnitine chloride |
| | 1.5 mg diheptanoylphosphatidylcholine |

Administration Route: i.d.
   Group 4 (n=1)
Vol (BMP-6-MAG-3)=200 μL
Vol (acetate buffer 0.1 M, pH 3.0)=400 μL
Total Vol=600 μL
Addition of enhancer: 1.5 mg diheptanoylphosphatidylcholine
Administration Route: i.d.
   Group 5 (n=1)
Vol (BMP-6-MAG-3)=200 μL
Vol (acetate buffer 20 mM, pH 4.0)=400 μL
Total Vol=600 μL
Administration Route: Intravenously (i.v.)

The animal in Group 1 received BMP-6 at a dose of 100 μg/kg in standard acetate buffer (20 mM, pH 4.0) injected intraduodenally (i.d.). Animals in Groups 2, 3, and 4 received BMP-6 at a dose of 100 μg/kg in acetate buffer (0.1 M, pH 3.0) and different combination of enhancers (e.g., taurodeoxycholic acid sodium, DL-lauroylcarnitine chloride, and/or diheptanoylphosphatidylcholine) injected into the duodenum. The animal in Group 5 received BMP-6 at a dose of 100 μg/kg in acetate buffer (20 mM, pH 4.0) injected intravenously (i.v.).

Intraduodenal (i.d.) application. Animals were anesthetized with thyopenthal and were subjected to abdominal surgery. After revealing of abdominal organs and isolation of the duodenum, BMP-6 labeled with 99mTc was injected with a syringe and needle directly into duodenum. Animals were sacrificed 60 minutes after surgery, and blood and all organs were taken for measurement.

Measurement of radioactivity with gamma counter. All samples were measured for the amount of radioactivity with a gamma counter and were expressed as counts per minute (cpm). The results were expressed as a percentage of measured radioactivity in the blood of an intraduodenally injected animal compared to radioactivity measured in the blood of an intravenously injected animal (Group 5) at the same time following surgery. All values were corrected in dependence of the half-life factor.

Results

Results are shown in FIG. 17. An animal, such as Animal 1, that received BMP-6 alone without lowering of pH or addition of enhancers had in its blood 1.6% of the BMP-6 measured in blood of an intravenously applied animal. Animals receiving BMP-6 with the acetate buffer (0.1 M, pH 3.0) and 1 mg of taurodeoxycholic acid sodium and 1 mg of DL-lauroylcarnitine chloride had increased duodenal absorption of BMP-6 to 35% (an average of animals in Group 2 is shown in FIG. 17) of intravenous dose suggesting that those animals absorbed 35% of the applied BMP-6. Animals that received BMP-6 with the acetate buffer (0.1 M, pH 3.0) and 1.5 mg of diheptanoylphosphatidylcholine in various combinations had absorption of approximately 6% of an intravenous dose (see, Groups 3 and 4 in FIG. 17).

### Example 6

### In vitro Duodenal Absorption of BMP-6 Labeled with 99mTc by Everted Gut Sac Technique

BMP-6 labeling. Mature BMP-6 was chelated with mercaptoacetyl-3-glycin (MAG3). BMP-6-MAG3 complex was labeled with radioactive 99mTechnetium-pertechnetate (99mTc). Chromatography revealed that more than 97% of 99mTc was ligated to the complex.

Everted gut sac technique. Sprague-Dawley rats were sacrificed, and the first 10 cm of the intestine distal to the pyloric valve was dissected free. The tissue was immediately rinsed with an isotonic solution of sodium chloride. Following the rinse, much of the mesentery was trimmed free, and the intestine everted (mucosal side became external and serosal side became internal) in a manner such that the distal end of the segment remained tied to an everting rod. The intestine was then tied with a ligature just distal to the pyloric valve and rinsed as before. It was then blotted, trimmed to a length of 5.5 cm and filled with 0.5 ml of incubation medium using a syringe fitted with a blunt needle. The sac was then placed in a 25 mL Erlenmeyer flask containing 10 mL of the same incubation medium and incubated at 37° C. for 90 minutes. Oxygen was continuously bubbled through the incubation medium throughout the experiment.

Incubation Medium

Two incubation media were employed:
Medium 1: 154 mmol/L NaCl, 16.6 mmol/L glucose
Medium 2: 125 mM NaCl, 10 mM glucose, 30 mM Tris-Cl buffer (pH 7.4), 0.25 mM CaCl₂
   Protocol

BMP-6 labeled with 99mTc (8.64 μg) was dissolved in 70 μL of acetate buffer (20 mM, pH 4.0) was added to 10 mL of incubation medium at the external, mucosal side of the gut, since the in vivo absorption occurs from the mucosal to the serosal side of the gut. Acetate buffer (70 μL, 20 mM, pH 4.0) alone was added in the incubation medium on the mucosal side of the gut, which was used as a control. The in vitro everted gut systems were:
1. 8.64 μg of BMP-6 labeled with 99mTc+70 μL of acetate buffer (20 mM, pH 4.0)+10 ml Medium 1.
2. 8.64 μg of BMP-6 labeled with 99mTc+70 μL of acetate buffer 20 mM, (pH 4.0)+10 ml Medium 2.
3. Control: 70 μL of acetate buffer 20 mM (pH 4.0)+10 ml Medium 1

Measurement of radioactivity with gamma counter. After 90 minutes of incubation, external and internal media were measured for the amount of radioactivity with gamma counter and were expressed as counts per minute (cpm). The results were also expressed as a percentage of measured radioactivity in the internal medium on the serosal side compared to radioactivity measured in the external medium at the mucosal side. All values were corrected in dependence of the half-life factor.

Measurement of chemical parameters. Glucose and lactic acid were determined calorimetrically, and Na, Cl, and Ca by using commercial kits.

Results

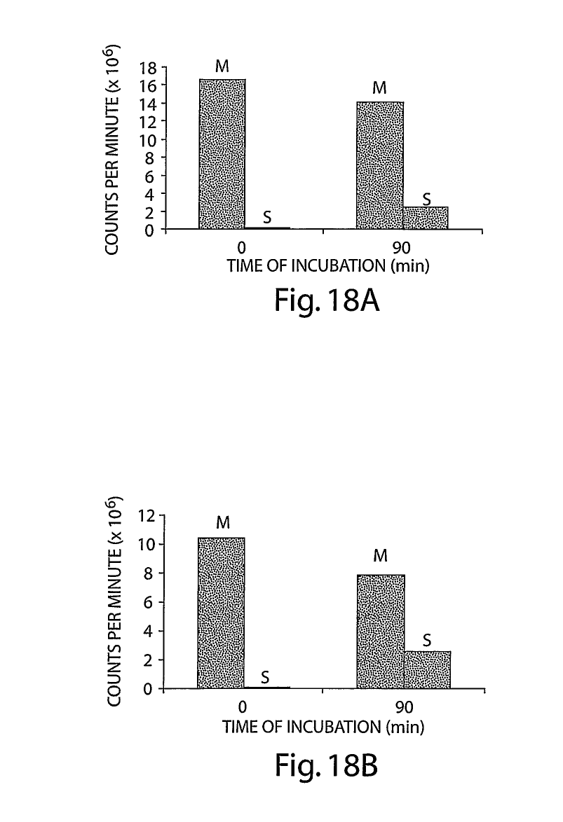

The results are shown as bar graphs in FIGS. 18A (Medium 1 data) and 18B (Medium 2 data). With respect to Medium 1 (no buffering system), after 90 minutes of incubation, 17.7% of the labeled BMP-6 was transferred from the mucosal (M) to the serosal (S) surface of the gut (see, 90 min data in FIG. 18A). In contrast, using Medium 2 (containing pH 7.4 buffer and calcium), 32.2% of labeled BMP-6 was transferred from the mucosal to the serosal surface of the gut in the medium (see, 90 min data in FIG. 18B). An average of 99.8% of glucose in the starting solution was metabolized during the incubation period of 90 minutes. Lactic acid production was increased 4-fold in the everted gut systems containing BMP-6 as compared to the control system suggesting that transference of BMP-6 required production of more energy (see, Table 2, below).

TABLE 2
Values of chemical parameters after 90 minutes of incubation.

| | BMP-6 + | | BMP-6 + | | | |
|---|---|---|---|---|---|---|
| | Medium 1 | | Medium 2 | | Control | |
| | serosal | mucosal | serosal | serosal | serosal | mucosal |
| Glucose* | 0 | 0.4 | 0 | 0.1 | 0 | 0.1 |
| Lactic | 1.4 | 5.91 | 0.46 | 1.61 | 0.55 | 1.83 |
| acid* | | | | | | |
| Na* | 129 | 149 | 97 | 105 | 141 | 149 |
| Cl* | 122 | 135 | 112 | 125 | 135 | 143 |
| Ca* | | | 1.19 | 1.43 | | |

*All results expressed in mmol/L.

### Example 7

### Degradation of BMP-6 by Specific Gastric and Intestinal Enzymes

The above studies showed that BMP-6 can be effectively absorbed into the body when present in the duodenum. This study examined the sensitivity of BMP-6 to gastric and intestinal enzyme degradation.

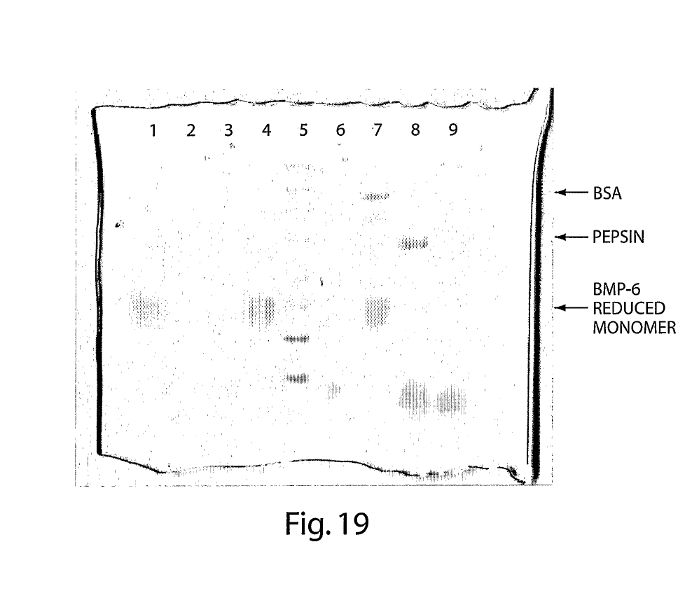

Sensitivity to pepsin proteolytic degradation. BMP-6 (10 μg) was incubated with 0, 1, 5, or 10 μL of pepsin (2500-3500 IU/mg). Bovine serum albumin (BSA) (5 μg) was used as a positive control for pepsin degradation activity. The digestion reaction products were examined by electrophoresis on polyacrylamide gels under reducing (dithiothreitol, “DTT”) conditions, which permit the tracking of the BMP-6 monomer. The gels were stained with Coomassie blue for visualization. The reaction products of BMP-6 incubated in the presence of 0, 10, 5, and 1 μL of pepsin are shown in lanes 1-4, respectively, of the gel in FIG. 19. Lanes 6 and 7 of FIG. 19 contain the reaction products of BMP-6 and BSA incubated in the presence of 5 and 1 μL of pepsin, respectively. Lanes 8 and 9 contain of FIG. 19 contain the reaction products of BSA incubated in the presence of 5 and 1 μL of pepsin, respectively. Lane 5 of FIG. 19 contains molecular weight standards.

As shown in FIG. 19, pepsin degraded both BSA and BMP-6 (see, e.g., BMP-6 degradation in lanes 1-4 and BSA degradation in lanes 8 and 9, of FIG. 19). In the presence of BSA, less BMP-6 was degraded than when BMP-6 was present alone in the reaction mixture (e.g., compare lanes 6 and 7 with lanes 3 and 4 of FIG. 19). Pepsin degradation of BSA appeared to first yield a shorter polypeptide of about 47 kilodalton (kDa) and, eventually, smaller fragments. The results showed that pepsin degraded BMP-6 rapidly in a dose specific manner (lanes 1-4 of FIG. 19). Sensitivity to trypsin and chymotrypsin proteolytic degradation. In a manner similar to testing for sensitivity to pepsin degradation, above, BMP-6 (10 μg) or BSA (5 μg), as a negative control, was incubated with intestinal enzymes, trypsin (6,000-12,000 μg/mg) or chymotrypsin (40-60 IU/mg). Digestion products were analyzed by electrophoresis on polyacrylamide gels under reducing conditions, as above. The results are shown in the gel in FIG. 20 (lane 4, molecular weight standards). Reaction products of BMP-6 incubated in the presence of 0, 1, and 0.2 μL trypsin are shown in lanes 1, 2, and 3, respectively, of the gel in FIG. 20. Reaction products of BMP-6 incubated in the presence of 0.5 and 0.2 μL chymotrypsin are shown in lanes 5 and 6, respectively. Lane 7 of FIG. 20 shows reaction products of BMP-6 and BSA incubated in the presence of 0.2 μL trypsin, and lane 8 shows reaction products of BMP-6 and BSA incubated in the presence of 0.2 μL chymotrypsin. Lane 9 of FIG. 20 shows reaction products of BSA incubated in the presence of 0.2 μL trypsin, and lane 10 shows the reaction products of BSA incubated in the presence of 0.2 μL chymotrypsin.

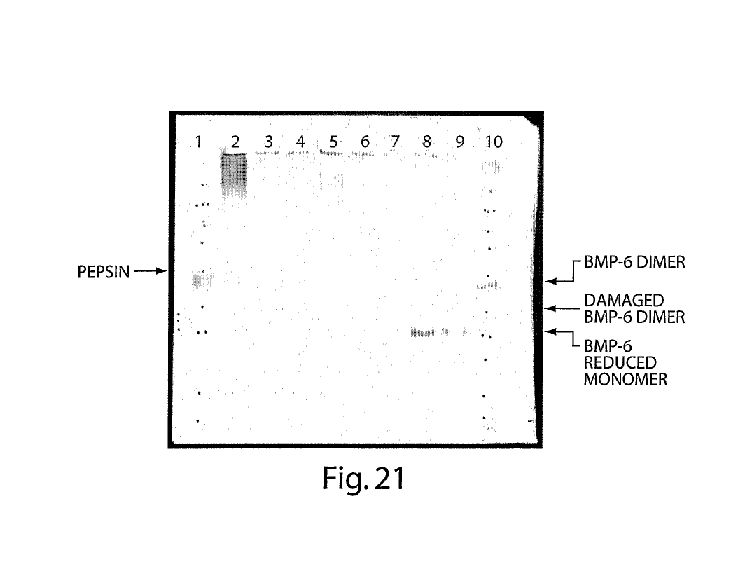

Trypsin only caused a slight truncation of BMP-6 monomeric polypeptide (see, lanes 2 and 3 of FIG. 20), whereas chymotrypsin was capable of effectively degrading BMP-6 in a dose specific manner (see, lanes 5 and 6 of FIG. 20). Lower amounts of chymotrypsin (e.g., 0.2 μL) indicated an initial truncation of the BMP-6 monomer (lane 6 of FIG. 20). BSA was resistant to degradation by either trypsin (see, lanes 7 and 9 of FIG. 20) or chymotrypsin (see, lanes 8 and 10 of FIG. 20). Gastric juice digestion of BMP-6. Separate samples of gastric juice were collected from two fasted, Sprague-Dawley rats. BMP-6 (0.5 μg) was incubated with varying amounts (1 μL, 10 μL) of the gastric juice samples. Digestion products were analyzed by Western immunoblotting electrophoresed under reducing (+DTT) conditions to track BMP-6 monomer or under non-reducing (no DTT) conditions to track BMP-6 dimer. Anti-BMP-6 antibody (SV-17, rabbit pooled serum) was used to detect BMP-6. Results are shown in Western immunoblot of FIG. 21 (lanes 1 and 10 show standard molecular weight markers). Reaction products of BMP-6 incubated in the presence of 10, 1, and 1 μL of gastric juice from animal 1 are shown in lanes 2, 3, and 4 (no DTT), respectively, of FIG. 21, and reaction products of BMP-6 incubated in the presence of 10, 1, and 1 μL of gastric juice from animal 2 are shown in lanes 5, 6, and 7 (no DTT), respectively. Reaction products of BMP-6 incubated in the presence of 10, 1, and 1 μL heat-inactivated (90° C., 1 minute) gastrict juice are shown in lanes 8, 9, and 10 (no DTT and includes molecular weight standards).

Both samples of rat gastric juice degraded BMP-6 as shown by loss of BMP-6 dimer (see, lanes 4 and 7 in FIG. 21) or monomer (see, e.g., lanes 2, 3, 5, and 6 in FIG. 21). Under non-reducing conditions, pepsin in the gastric juice samples migrates at a position of 35 kDa, which is similar to migration position of the BMP-6 dimer (e.g., lanes 4 and 7 in FIG. 21). The BMP-6 monomer was only detected when the gastric juice samples were heated (90° C., 1 min) sufficiently to destroy the proteolytic activity (see, lanes 8 and 9 of FIG. 21). Gastric juice sample of animal 1 was more active than the gastric juice sample of animal 2 as evidenced by the appearance in reaction products of BMP-6 and gastric juice from animal 2 of a slightly truncated (i.e., partially digested) BMP-6 dimer species migrating at about 28 kDa (see, lane 7 of FIG. 21) that was detected with a specific anti-BMP-6 antibody (N-19, Santa Cruz Biotechnology, Inc., Santa Cruz, Calif.).

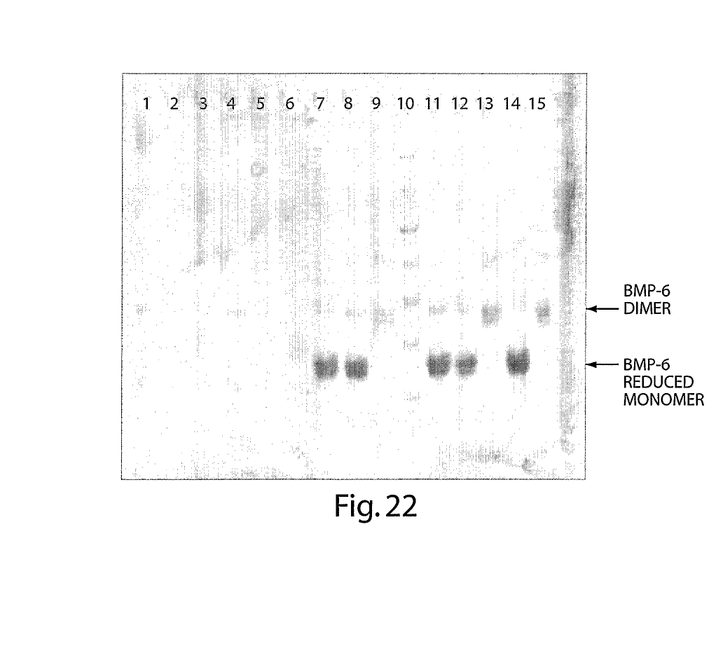

Gastric juice digestion in the presence and absence of the pepsin inhibitor pepsinostreptin. BMP-6 (0.5 μg) was incubated with varying amounts (1 μL, 10 μL) of the gastric juice samples, as described above, but in the presence and absence of the pepsin inhibitor pepsinostreptin (Roche Diagnostics, Corp., Indianapolis, Ind.). The reaction products were analyzed as described above by Western immunoblotting of gels run under reducing and non-reducing (no DTT) conditions to track BMP-6 monomer and dimer, respectively. Results are shown in Western immunoblot of FIG. 22 (lane 10 shows standard molecular weight markers, lane 14 contains BMP-6 monomer; lane 15 (no DTT) contains BMP-6 dimer). Reaction products of BMP-6 incubated with 10, 1, and 1 μL of gastric juice from animal 1 are shown in lanes 1, 2, and 3 (no DTT), respectively, of FIG. 22, and reaction products of BMP-6 incubated with 10, 1, and 1 μL of gastric juice from animal 2 are shown in lanes 4, 5, 6 (no DTT). Reaction products of BMP-6 incubated in the presence of the pepsin inhibitor pepsinostreptin and 10, 1, and 1 μL of gastric juice are shown in lanes 7, 8, and 9 (no DTT), respectively. Reaction products of BMP-6 incubated in the presence of 10, 1, 1 μL of heat-inactivated gastric juice are shown in lanes 11, 12, and 13, respectively.

Pepsinostreptin completely inhibited the proteolytic activity of the gastric juice on BMP-6, as shown by preservation of the BMP-6 monomer under reducing conditions (see, lanes 7 and 8 of FIG. 22) or of the BMP-6 dimer under non-reducing conditions (see, lane 9 of FIG. 22). The result was similar to the negative control containing BMP-6 incubated with heat-inactivated gastric juice, as described above (see, lanes 11-13 of FIG. 22).

The above data indicate that pepsin is the primary source of proteolytic degradation of BMP-6 in the stomach.

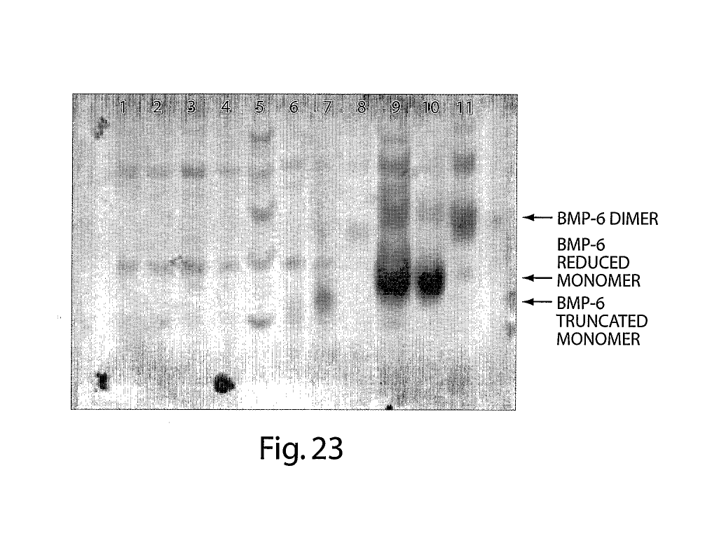

Duodenal juice digestion of BMP-6. Samples of duodenal juice were collected from two fasted Sprague-Dawley rats. BMP-6 (0.5 μg) was incubated with varying amounts (1 μL, 3 μL) of the duodenal juice, and digestion products analyzed by Western blotting of polyacrylamide gels under reducing and non-reducing conditions (no DTT), as described above. Results are shown in the Western immunoblot of FIG. 23. Dimer of BMP-6 is shown under non-reducing conditions in lane 11 (no DTT) of FIG. 23, and the BMP-6 monomer is shown under reducing conditions (+DTT) in lane 10 of FIG. 23. Standard molecular weight markers are shown in lane 5 of FIG. 23. Reaction products of BMP-6 incubated with 3 and 1 μL of duodenal juice from animal 1 are shown in lanes 1 and 2, respectively, of FIG. 23, and the reaction products of BMP-6 incubated with the 3 and 1 μL of duodenal juice from animal 2 are shown in lanes 3 and 4, respectively. Lanes 6, 7, and 8 (no DTT) of FIG. 23 show the reaction products of BMP-6 incubated in the presence of acetate buffer (pH 3) and 3, 1, and 1 μL of duodenal juice. Lane 9 shows the products of BMP-6 incubated with 1 μL of heat-inactivated duodenal juice.

Duodenal juice from both animals effectively degraded BMP-6 in a dose specific manner (see, lanes 1-4 of FIG. 23). Incubating reactions in the presence of pH 3 acetate buffer provided partial protection from degradation by duodenal juice (see, lanes 6-8 of FIG. 23). The proteolytic activity of the duodenal juice could be destroyed by heating (see, lane 9 of FIG. 23).

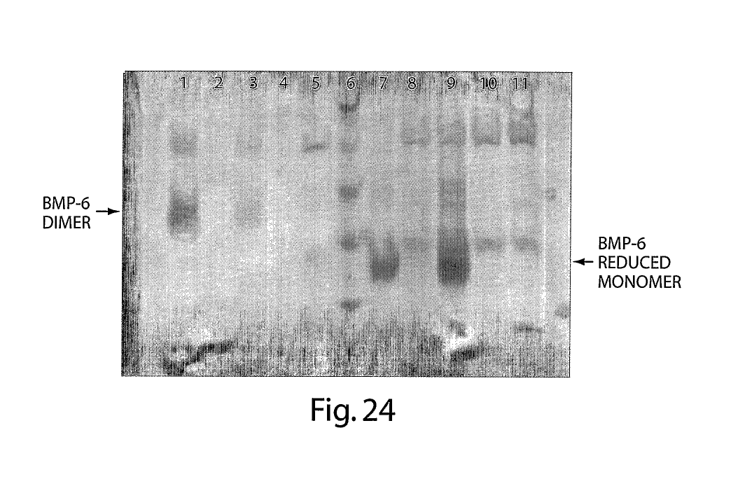

The effect of protease inhibitors on duodenal proteolytic activity. The effect of several protease inhibitors on the ability of duodenal juice to degrade BMP-6 was tested. BMP-6 was incubated with duodenal juice as described above, except in the presence and absence of various other protease inhibitors. Reaction products were electrophoresed on polyacrylamide gels under non-reducing conditions (no DTT) to track BMP-6 dimer and under reducing conditions (+DTT) to track BMP-6 monomer as described above. The gel was then analyzed by Western immunoblotting. Results are shown in the Western immunoblot of FIG. 24 (lane 1 shows BMP-6 dimer, lane 7 shows BMP-6 monomer, and lane 6 shows standard molecular weight markers). Lanes 1-5 of FIG. 24 show results under non-reducing condition (no DTT), and lanes 9-11 show results under reducing condition (+DTT). Reaction products of BMP-6 incubated with 1 μL duodenal juice are shown in lanes 2 and 8, respectively. Reaction products of BMP-6 incubated with 1 μL duodenal juice and 1 μL of chymostatin (chymotrypsin-specific inhibitor, 1 μL, Roche Diagnostics Corp., Indianapolis, Ind., U.S.) are shown in lanes 3 and 9, respectively. Reaction products of BMP-6 incubated with 1 μL duodenal juice and 1 μL of soybean trypsin inhibitor are shown in lanes 4 and 10, and reaction products of BMP-6 incubated with 1 μL duodenal juice and 1 μL of aprotinin (also called bovine pancreatic trypsin inhibitor or BPTI, a broad spectrum protease inhibitor, Roche Diagnostics Corp., Indianapolis, Ind.) are shown in lanes 5 and 11. The results from FIG. 24 show that only chymostatin protected proteolytic degradation of BMP-6 as shown by preservation of BMP-6 dimer (lane 3) or BMP-6 monomer (lane 9).

The data indicate that chymotrypsin is the primary source of BMP-6 proteolytic degradation in the duodenum.

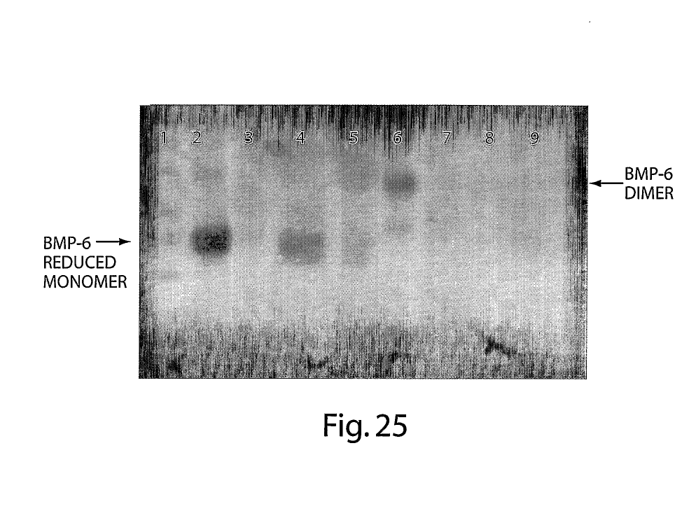

The effect of altering pH on duodenal chymotrypsin-mediated degradation of BMP-6. The above studies indicate that chymotrypsin is the enzyme primarily responsible for degradation of BMP-6 in incubations with duodenal juice. The effect of pH on the proteolytic activity of the duodenal chymotrypsin was also tested. BMP-6 was incubated with duodenal juice as described above, except that incubations were carried out at various pH values. Reaction products were electrophoresed on polyacrylamide gels that were subsequently analyzed by Western immunoblotting as described above. Results are shown in the Western immunoblot of FIG. 25 (lane 1 shows molecular weight standards, lane 2 shows BMP-6 monomer alone, lane 6 shows BMP-6 dimer alone). Lanes 2-5 of FIG. 25 were run under reducing conditions (+DTT) to track BMP-6 monomer, and lanes 6-9 were run under non-reducing conditions (no DTT) to track BMP-6 dimer. Reaction products of BMP-6 incubated with duodenal juice at pH 7 are shown in lanes 3 and 7 of FIG. 25. Reaction products from analogous incubations carried out at pH 4, are shown in lanes 4 and 8, and reaction products from incubations carried out at pH 5 are shown in lanes 5 and 9. The results clearly show that proteolytic degradation of BMP-6 (monomer and dimer) by duodenal juice progressively decreased in reaction mixtures as the pH was decreased from 7 (lanes 3 and 7) to 5 (lanes 5 and 9) to 4 (lanes 4 and 8), consistent with the known pH sensitivity of duodenal chymotrypsin.

### Example 8

### Effect in vivo of Oral and Duodenal Application of BMP-6 and Enzyme Inhibitors on Bones in Aged Ovariectomized Rats

This study was conducted to determine whether BMP-6, when protected from degradation by gastric pepsin and duodenal chymotrypsin, is effectively absorbed along the alimentary canal to restore and improve bone mineral density (BMD) in a rat model of osteoporosis.

Six month-old Sprague-Dawley rats were ovariectomized (OVX) and left for 6 months to permit loss of BMD. Animals received BMP-6 by intraduodenal (i.d.) administration or by gastric tube. Therapy began 6 months following ovariectomy at the age of 12 months and continued for 3 weeks according to the following treatment Groups:

| | |
|---|---|
| Group 1. | SHAM (n = 15) |
| Group 2. | OVX control, acetate buffer, pH 3.5, i.d. (n = 10) |
| Group 3. | OVX treated with 500 μg/kg BMP-6, 20 μg chymostatin, |
| | 20 μg aprotinin, pH 7.0, injected i.d., once per week (n = 14) |
| Group 4. | OVX treated with 500 μg/kg BMP-6, 20 μg chymostatin, |
| | 20 μg aprotinin, pH 3.5, injected i.d., once per week (n = 14) |
| Group 5. | OVX treated with 300 μg/kg (b.w.) BMP-6, |
| | 50 μg chymostatin, 50 μg aprotinin, 50 μg pepstatin, |
| | pH 3.5, per os delivery with gastric tube, three times |
| | per week (n = 14) |

Bone Mineral Density (BMD) in vivo

Based on protease inhibition studies described above, the presence of aprotinin in this study was considered to not play a significant role in protecting BMP-6 from gastric or duodenal proteolytic degradation. In vivo BMD was monitored at the beginning of therapy and 3 weeks following. After 3 weeks of therapy, all animals treated with BMP-6 showed higher BMD values of hind limbs as compared to OVX animals.

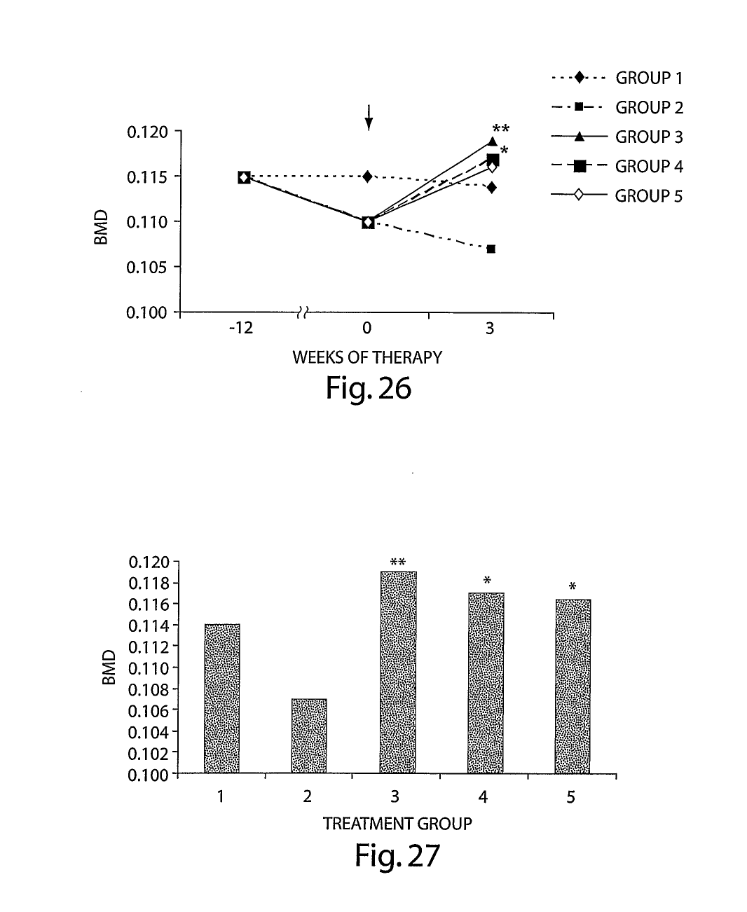

The changes in BMD for treatment Groups 1-5 are shown in FIGS. 26 and 27. FIG. 26 shows the results over the course of the three-week treatment period. FIG. 27 compares the final BMD values attained for animals of the various treatment Groups by the end of the treatment period. Similar results were observed in treatment Groups 3, 4, and 5. Group 4 animals that received BMP-6 with protease inhibitors at pH 3.5, i.d., had BMD hind limb values that were 7.1% higher than Group 2 OVX control animals receiving acetate buffer alone. Groups 3, 4, and 5 animals also showed somewhat higher BMD values than Group 1 SHAM animals. At 3 weeks, all groups that received BMP-6 in combination with protease inhibitors (i.e., Groups 3, 4, 5) showed statistically significant increases in BMD values of hind limbs compared to Group 2 OVX animals that received acetate buffer alone, e.g., for Group 3, P<0.005; for Groups 4 and 5, P<0.05 (see, FIGS. 26 and 27).

The data indicate that when proteolytical activities of gastric pepsin and duodenal chymotrypsin are inhibited, orally administered BMP-6 is effectively absorbed along the alimentary canal to significantly restore and even increase bone mineral density in critical bones to effectively treat osteoporosis.

### Example 9

### Effect of Duodenal Application of other Bone Morphogenetic Proteins on Bone Formation in Subcutaneous Bone Pellet (Matrix)

This study used the subcutaneously implanted bone pellet (matrix) assay described above in Example 4 to examine the effect of duodenal application of BMP-7 and cartilage-derived morphogenetic protein-2 (CDMP-2, BMP-13) on bone formation. In this assay, a demineralized and extracted bone matrix is implanted subcutaneously as a surrogate marker of bone formation.

Bone pellets. Donors for bone pellet preparation were Sprague-Dawley rats 20 weeks old. After sacrifice, diaphyses of femurs and tibias were taken for making the pellet. Bones were prepared with addition of chloric acid and urea. In subcutaneously implanted bone pellet there is no spontaneous formation of new bone.

Animals and treatment protocol. Sprague-Dawley rats weighing approximately 200 g were subjected to surgery. Bone pellets were implanted subcutaneously into the axillar region. Animals were divided into the following groups, with four pellets implanted per group

| | |
|---|---|
| 1. | Control (n = 4) |
| 2. | CDMP-2 (BMP-13), 500 μg/kg + acetate buffer (20 mM, pH = 3.5), |
| | 20 μg chymostatin, 20 μg aprotinin, i.d. (500 μL) (n = 3) |
| 3. | BMP-7, 500 μg/kg + acetate buffer (20 mM, pH = 3.5), 20 μg |
| | chymostatin, 20 μg aprotinin, i.d (500 μL) (n = 3) |

Animals were injected directly into the duodenum twice at 6 hours (h) and 25 h following implantation of bone pellet. Animals were sacrificed 15 days after surgery, and bone pellets were taken for histology.

Histology. Bone pellets were fixed in 70% alcohol, decalcified, and embedded in paraffin. Sections were stained with toluidine blue. Pellets were considered positive in the presence of new bone formation.

Results

As shown in Table 3, below, animals receiving CDMP-2 (BMP-13) and BMP-7, i.d., showed new bone formation in subcutaneous bone pellets, suggesting that both proteins can pass the gastrointestinal system in sufficient amount for systemic action on bone formation when protected from intestinal chymotypsin degradation. Control animals did not show signs of osteoinduction in bone pellets.

TABLE 3

| | New bone formation |
|---|---|
| Treatment Group | in implanted pellets |
| Control | − |
| CDMP-2 (500 μg/kg) | +++ |
| BMP-7 (500 μg/kg) | +++ |

−, no new bone formation detected
+ to +++, showing the degree of new bone formation

All patents, applications, and publications cited in the above text are incorporated herein by reference.

Other variations and embodiments of the invention described herein will now be apparent to those skilled in the art without departing from the disclosure of the invention or the coverage of the claims to follow.

1. A method of treating a metabolic bone disease that is characterized by a loss of bone mass in an individual comprising orally administering to the individual a formulation comprising:
an osteoinductive bone morphogenetic protein (BMP) or functionally equivalent osteoinductive protein,
an agent to prevent or inhibit proteolytic activity of intestinal chymotrypsin,
optionally, an agent to prevent or inhibit proteolytic activity of gastric pepsin,
optionally, an agent to prevent or inhibit proteolytic activity of intestinal trypsin, and
optionally, an absorption enhancer.

2. The method according to claim 1, wherein said osteoinductive BMP is selected from the group consisting of BMP-2, BMP-6, BMP-7, BMP-9, BMP-12, BMP-13, and combinations thereof.

3. The method according to claim 2, wherein said osteoinductive BMP is a purified naturally occurring protein or a purified recombinant protein.

4. The method according to claim 1, wherein said agent to prevent or inhibit proteolytic activity of intestinal chymotrypsin is selected from the group consisting of a pH lowering agent, a chymotrypsin-specific inhibitor, and combinations thereof.

5. The method according to claim 4, wherein said pH lowering agent is a buffer selected from the group consisting of acetate, succinate, lactate, citrate, isocitrate, ascorbate, oxaloacetate, oxalate, malate, fumarate, 2-ketoglutarate, glutarate, pyruvate, glycerate, and combinations thereof.

6. The method according to claim 4, wherein said chymotrypsin-specific inhibitor is selected from the group consisting of chymostatin, Z-L-phe chloromethyl ketone, α2-antiplasmin, aprotinin, 6-aminohexanoic acid, α1-antitrypsin, 4-(2-aminoethyl)benzenesulfonyl fluoride hydrochloride, bromoenol lactone, diisopropyl fluorophosphate, ecotoin, N-acetyl-eglin C, gabexate mesylate, leupeptin trifluoroacetate salt, N-p-tosyl-L-phenylalanine chloromethyl ketone, soybean trypsin-chymotrypsin inhibitor, and combinations thereof.

7. The method according to claim 1, wherein said agent to prevent or inhibit proteolytic activity of intestinal chymotrypsin is prepared from wheat, rice, oat, soybean, and combinations thereof.

8. The method according to claim 1, wherein said agent to prevent or inhibit proteolytic activity of gastric pepsin, when present, is selected from the group consisting of an enteric coating, a gastric pH regulating agent, a pepsin-specific inhibitor compound, and combinations thereof.

9. The method according to claim 8, wherein said enteric coating comprises a compound selected from the group consisting of cellulose acetate phthlate (CAP), cellulose acetate trimellite, hydroxypropylmethylcellulose phthlate, hydroxpropylmethyl cellulose acetate succinate, polyvinyl acetate phthlate, methacrylic acid copolymers, ethyl acrylate copolymers, and combinations thereof.

10. The method according to claim 8, wherein said gastric pH regulating agent is selected from the group consisting of an antacid, a compound that blocks histamine H2 receptors, a proton pump inhibitor, and combinations thereof.

11. The method according to claim 10, wherein said antacid is selected from the group consisting of calcium carbonate, sodium bicarbonate, aluminum hydroxide, magnesium hydroxide, aluminum carbonate gel, and combinations thereof.

12. The method according to claim 10, wherein said compound that blocks histamine H2 receptors is selected from the group consisting of cimetidine, famotidine, nizatidine, ranitidine, and combinations thereof.

13. The method according to claim 10, wherein said proton pump inhibitor is selected from the group consisting of lansoprazole, omeprazole, pantoprazole, abeprazole, and combinations thereof.

14. The method according to claim 8, wherein said pepsin-specific inhibitor compound is selected from the group consisting of pepstatin A, pepsinostreptin, and combinations thereof.

15. The method according to claim 1, wherein said agent to inhibit proteolytic activity of intestinal trypsin, when present, is selected from the group consisting of aprotinin, α2-antiplasmin, antithrombin III, α1-antitrypsin, antipain, 4-(2-aminoethyl)benzenesulfonyl fluoride hydrochloride, p-aminobenzamidine dihidrochloride, bdellin, benzamidine hydrochloride, diisopropyl fluorophosphate, 3,4-dichloroisocoumarin, ecotin, gabexate mesylate, leupeptin, α2-macroglobulin, phenylmethylsulfonyl fluoride, N-α-p-tosyl-L-phenylalanine chloromethyl ketone, trypsin-chymotrypsin inhibitor, and combinations thereof.

16. The method according to claim 1, wherein said agent to prevent or inhibit proteolytic activity of intestinal chymotrypsin is released from said formulation in the duodenum and inhibits proteolytic activity of intestinal chymotrypsin for a period of time sufficient to permit absorption of said osteoinductive BMP or functionally equivalent osteoinductive protein from the intestines into the bloodstream.

17. The method according to claim 1, wherein said absorption enhancer, when present, is a surface active agent selected from the group consisting of an anionic agent that is a cholesterol derivative, a cationic surface active agent, a non-ionic surface active agent, and combinations thereof.

18. The method according to claim 17, wherein said anionic agent that is a cholesterol derivative is a bile acid.

19. The method according to claim 18, wherein said bile acid is selected from the group consisting of cholic acid, deoxycholic acid, taurocholic acid, taurodeoxycholic acid, fusidic acid, glycholic acid, dehydrocholic acid, lithocholic acid, ursocholic acid, ursodeoxycholic acid, and combinations thereof.

20. The method according to claim 17, wherein said a cationic surface active agent is selected from the group consisting of an acylcarnitines, an acylcholine, a lauroylcholine, a cetyl pyridinium chloride, a cationic phospholipid, and combinations thereof.

21. The method according to claim 17, wherein said non-ionic surface active agent is selected from the group consisting of a polyoxyethylene ether, a p-t-octyl phenol poloxyethylene, a nonylphenoxypoloxyethylene, a polyoxyethylene sorbitan ester, and combinations thereof.

22. The method according to claim 1, wherein the formulation is administered to the individual through the mouth, through the intestines by injection, or rectally.

23. A formulation for the oral delivery of an osteoinductive BMP to an individual comprising:
an osteoinductive bone morphogenetic protein (BMP),
an agent to prevent or inhibit proteolytic activity of intestinal chymotrypsin,
optionally, an agent to prevent or inhibit proteolytic activity of gastric pepsin,
optionally, an agent to prevent or inhibit proteolytic activity of intestinal trypsin, and
optionally, an absorption enhancer.

24. The formulation according to claim 23, wherein said osteoinductive BMP is selected from the group consisting BMP-2, BMP-6, BMP-7, BMP-9, BMP-12, BMP-13, and combinations thereof.

25. The formulation according to claim 24, wherein said osteoinductive BMP is a purified naturally occurring protein or a purified recombinant protein.

26. The formulation according to claim 23, wherein said agent to prevent or inhibit proteolytic activity of intestinal chymotrypsin is selected from the group consisting of a pH lowering agent, a chymotrypsin-specific inhibitor, and combinations thereof.

27. The formulation according to claim 26, wherein said pH lowering agent is a buffer selected from the group consisting of acetate, succinate, lactate, citrate, isocitrate, ascorbate, oxaloacetate, oxalate, malate, fumarate, 2-ketoglutarate, glutarate, pyruvate, glycerate, and combinations thereof.

28. The formulation according to claim 26, wherein said chymotrypsin-specific inhibitor is selected from the group consisting of chymostatin, Z-L-phe chloromethyl ketone, α2-antiplasmin, aprotinin, 6-aminohexanoic acid, α1-antitrypsin, 4-(2-aminoethyl)benzenesulfonyl fluoride hydrochloride, bromoenol lactone, diisopropyl fluorophosphate, ecotoin, N-acetyl-eglin C, gabexate mesylate, leupeptin trifluoroacetate salt, N-p-tosyl-L-phenylalanine chloromethyl ketone, soybean trypsin-chymotrypsin inhibitor, and combinations thereof.

29. The formulation according to claim 23, wherein said agent to prevent or inhibit proteolytic activity of intestinal chymotrypsin is prepared from wheat, rice, oat, soybean, and combinations thereof.

30. The formulation according to claim 23, wherein said agent to prevent or inhibit proteolytic activity of gastric pepsin, when present, is selected from the group consisting of an enteric coating, a gastric pH regulating agent, a pepsin-specific inhibitor compound, and combinations thereof.

31. The formulation according to claim 30, wherein said enteric coating comprises a compound selected from the group consisting of cellulose acetate phthlate (CAP), cellulose acetate trimellite, hydroxypropylmethylcellulose phthlate, hydroxpropylmethyl cellulose acetate succinate, polyvinyl acetate phthlate, methacrylic acid copolymers, ethyl acrylate copolymers, and combinations thereof.

32. The formulation according to claim 30, wherein said gastric pH regulating agent is selected from the group consisting of an antacid, a compound that blocks histamine H2 receptors, a proton pump inhibitor, and combinations thereof.

34. The formulation according to claim 32, wherein said antacid is selected from the group consisting of calcium carbonate, sodium bicarbonate, aluminum hydroxide, magnesium hydroxide, aluminum carbonate gel, and combinations thereof.

35. The formulation according to claim 32, wherein said compound that blocks histamine H2 receptors is selected from the group consisting of cimetidine, famotidine, nizatidine, ranitidine, and combinations thereof.

36. The formulation according to claim 32, wherein said proton pump inhibitor is selected from the group consisting of lansoprazole, omeprazole, pantoprazole, abeprazole, and combinations thereof.

37. The formulation according to claim 30, wherein said pepsin-specific inhibitor compound is selected from the group consisting of pepstatin A, pepsinostreptin, phenylmethylsulfonyl fluoride, and combinations thereof.

38. The formulation according to claim 23, wherein said intestinal trypsin-specific inhibitor compound, when present, is selected from the group consisting of aprotinin, α2-antiplasmin, antithrombin III, α1-antitrypsin, antipain, 4-(2-aminoethyl)benzene sulfonyl fluoride hydrochloride, p-aminobenzamidine dihidrochloride, bdellin, benzamidine hydrochloride, diisopropyl fluorophosphate, 3,4-dichloroisocoumarin, ecotin, gabexate mesylate, leupeptin, α2-macroglobulin, phenylmethylsulfonyl fluoride, N-α-p-tosyl-L-phenylalanine chloromethyl ketone, trypsin-chymotrypsin inhibitor, and combinations thereof.

39. The formulation according to claim 23, wherein said absorption enhancer is a surface active agent selected from the group consisting of an anionic agent that is a cholesterol derivative, a cationic surface active agent, a non-ionic surface active agent, and combinations thereof.

40. The formulation according to claim 39, wherein said anionic agent that is a cholesterol derivative is a bile acid.

41. The formulation according to claim 40, wherein said bile acid is selected from the group consisting of cholic acid, deoxycholic acid, taurocholic acid, taurodeoxycholic acid, fusidic acid, glycholic acid, dehydrocholic acid, lithocholic acid, ursocholic acid, ursodeoxycholic acid, and combinations thereof.

42. The formulation according to claim 39, wherein said a cationic surface active agent is selected from the group consisting of an acylcarnitines, an acylcholine, a lauroylcholine, a cetyl pyridinium chloride, a cationic phospholipid, and combinations thereof.

43. The formulation according to claim 39, wherein said non-ionic surface active agent is selected from the group consisting of a polyoxyethylene ether, a p-t-octyl phenol poloxyethylene, a nonylphenoxypoloxyethylene, a polyoxyethylene sorbitan ester, and combinations thereof.
